(19) United States
(12) Patent Application Publication
(10) Pub. No.: US 2021/0356467 A1
(43) Pub. Date: Nov. 18, 2021

(54) SYSTEMS AND METHOD FOR SCREENING SMALL MOLECULES OF INTEREST

(71) Applicant: TECHNOLOGY INNOVATION MOMENTUM FUND (ISRAEL) LIMITED PARTNERSHIP, Tel-Aviv (IL)

(72) Inventor: Gali PRAG, Tel-Aviv (IL)

(73) Assignee: Technology Innovation Momentum Fund (Israel) Limited Partnership, Tel-Aviv (IL)

(21) Appl. No.: 17/285,133

(22) PCT Filed: Oct. 16, 2019
(86) PCT No.: PCT/IL2019/051122

§ 371 Date: Apr. 14, 2021

Related U.S. Application Data
(60) Provisional application No. 62/746,001, filed on Oct. 16, 2018.

(51) Int. Cl. G01N 33/573

(52) U.S. Cl. CPC G01N 33/573; G01N 2333/9108; C12Y 203/02

(57) ABSTRACT

Bacterial systems for screening for small molecule agents are disclosed herein. Kits for carrying out the screening are also disclosed.

### RELATED APPLICATION

This application claims the benefit of priority of U.S. Provisional Application No. 62/746,001 filed on 16 Oct. 2018, the contents of which are incorporated herein by reference in their entirety.

### SEQUENCE LISTING STATEMENT

The ASCII file, entitled 79430 Sequence Listing.txt, created on 15 Oct. 2019, comprising 12,288 bytes, submitted concurrently with the filing of this application is incorporated herein by reference.

### FIELD AND BACKGROUND OF THE INVENTION

The present invention, in some embodiments thereof, relates to bacterial systems for screening small molecules of interest.

Ubiquitin (Ub) plays a pivotal role in numerous aspects of cellular processes. Therefore, aberrations in the Ub system are involved in a large number of pathologies, including various forms of cancer such as breast and colon cancer, neurodegenerative diseases such as Parkinson's and Alzheimer's diseases, and infectious diseases such as HIV and Ebola. Consequently, there is a critical need for a detailed understanding of the Ub system. Although there have been significant advances in understanding the ubiquitination process, much less is known about the downstream processes. These include substrate recognition by specific enzymatic interactions in the Ub system, and specific interactions between these enzymes and their substrates. In humans, for example, there are 2 E1 Ub-activating enzymes, 34 Ub-conjugating enzymes and more than 600 E3 Ub-ligases. These enzymes work on presumably several thousands of protein substrates, where specificity is mainly achieved by the E2:E3 and E3:Substrates interactions.

Another factor which impedes the researchers' efforts to fully characterize Ub cascades is the presence of deubiquitinating enzymes (DUBs) which rapidly reverse the ubiquitination signal. The half-life time of ubiquitylated proteins is thus extremely short. Specifically, it has been shown that about 100 DUBs that exist reverse the modification in a highly specific manner.

Background art includes Keren-Kaplan et al., The EMBO Journal (2012) 31, 378-390; Levin-Kravets et al., Nature Methods 13, pages 945-952, 2016; and Su et al., J Immunol 2006; 177; 7559-7566.

Additional background art includes WO2019/030759 and WO2018/029682.

### SUMMARY OF THE INVENTION

According to an aspect of the present invention there is provided a method of identifying an agent which mediates the binding of a first polypeptide to a second polypeptide comprising:

(a) contacting a bacterial cell which expresses said first polypeptide and said second polypeptide with the agent, wherein the bacterial cell outputs a detectable or selectable signal which correlates with the non-direct binding of the first polypeptide to the second polypeptide via the agent, wherein the non-direct binding of the first polypeptide to the second polypeptide via the agent brings about the ubiquitination of the first polypeptide or the second polypeptide; and

(b) measuring the level or the rate of accumulation of the detectable or selectable signal, wherein a change in the level as compared to the level in the absence of the agent, is indicative of an agent which mediates the binding of the first polypeptide to the second polypeptide.

According to an aspect of the present invention there is provided a method of identifying an agent which mediates the binding of a first polypeptide to a second polypeptide comprising:

(a) contacting a bacterial cell with the agent, wherein the bacterial cell outputs a detectable or selectable signal which correlates with the non-direct binding of the first polypeptide to the second polypeptide via the agent; and

(b) measuring the level or the rate of accumulation of the detectable or selectable signal, wherein a change in the level as compared to the level in the absence of the agent, is indicative of an agent which mediates the binding of the first polypeptide to the second polypeptide, wherein:

(i) the first polypeptide is a substrate for ubiquitination;

(ii) the second polypeptide is attached to ubiquitin;

(iii) the bacterial cell expresses a ubiquitinating enzyme which attaches the ubiquitin to the first polypeptide if the agent mediates the binding of the first polypeptide to the second polypeptide.

According to an aspect of the present invention there is provided a kit comprising:

(i) a first polynucleotide which encodes a first polypeptide fragment which is operably linked to a bacterial regulatory sequence, and a first cloning site, wherein a position of the first cloning site is selected such that upon insertion of a sequence which encodes a first test polypeptide into the first cloning site, following expression in a bacterial cell, a fusion protein is generated which comprises the first test polypeptide in frame with the first polypeptide fragment; and

(ii) a second polynucleotide comprising a second nucleic acid sequence encoding a second polypeptide fragment which is attached to ubiquitin, the second nucleic acid sequence being operably linked to a bacterial regulatory sequence, the second nucleic acid sequence comprising a second cloning site, wherein a position of the second cloning site is selected such that upon insertion of a sequence which encodes a second test polypeptide into the second cloning site, following expression in a bacterial cell, a fusion protein is generated which comprises the second test polypeptide in frame with the second polypeptide fragment;

wherein the first polypeptide fragment associates with the second polypeptide fragment to generate a reporter polypeptide dependent on ubiquitination of the first test polypeptide.

According to an aspect of the present invention there is provided a method of identifying an agent which mediates the binding of a ubiquitin E3 ligase to a test polypeptide comprising:

(a) contacting a bacterial cell which expresses the E3 ligase with the agent, wherein the bacterial cell outputs a detectable or selectable signal which correlates with the non-direct binding of the E3 ligase to the test polypeptide via the agent; and

(b) measuring the level or the rate of accumulation of the detectable or selectable signal, wherein a change in the level as compared to the level in the absence of the agent, is indicative of an agent which mediates the binding of the E3 ligase to the test polypeptide.

According to an aspect of the present invention there is provided a kit comprising:

(i) a first polynucleotide which encodes a first polypeptide fragment which is operably linked to a bacterial regulatory sequence, and a cloning site, wherein a position of the cloning site is selected such that upon insertion of a sequence which encodes a test polypeptide into the cloning site, following expression in a bacterial cell, a fusion protein is generated which comprises the test polypeptide in frame with the first polypeptide fragment;

(ii) a second polynucleotide comprising a second nucleic acid sequence encoding a second polypeptide fragment which is attached to ubiquitin, the second nucleic acid sequence being operably linked to a bacterial regulatory sequence, wherein the first polypeptide fragment associates with the second polypeptide fragment via a mediating agent to generate a reporter polypeptide dependent on ubiquitination of the test polypeptide; and

(iii) the mediating agent.

According to an aspect of the present invention there is provided a method of identifying an agent which mediates the binding of an E2 conjugating enzyme to an E3 ligase comprising:

(a) contacting a bacterial cell which expresses the E2 conjugating enzyme and the E3 ligase with the agent, wherein the bacterial cell outputs a detectable or selectable signal which correlates with the non-direct binding of the E2 conjugating enzyme to the E3 ligase via the agent, wherein the non-direct binding of the E2 conjugating enzyme to the E3 ligase via the agent brings about the ubiquitination of a protein of interest; and

(b) measuring the level or the rate of accumulation of the detectable or selectable signal, wherein a change in the level as compared to the level in the absence of the agent, is indicative of an agent which mediates the binding of the E2 conjugating enzyme to the E3 ligase.

According to some embodiments of the invention, the polypeptide is a ubiquitin E3 ligase.

According to some embodiments of the invention, the ubiquitin E3 ligase is a chimeric E3 ligase.

According to some embodiments of the invention, the second polypeptide is a substrate for the ubiquitin E3 ligase.

According to some embodiments of the invention, the first polypeptide is attached to ubiquitin.

According to some embodiments of the invention, the second polypeptide is not a ubiquitin E3 ligase.

According to some embodiments of the invention, the bacterial cell expresses at least one ubiquitin ligase.

According to some embodiments of the invention, the ubiquitin ligase comprises ubiquitin E3-ligase.

According to some embodiments of the invention, the ubiquitin E3-ligase is selected from the group consisting of CRBN, betaTrcP, VHL-ElonginB-ElonginC-Hif1 complex, Siah2, PARKIN, Smurf1, MDM2, BRCA1, MURF1, TRIM32, ITCH, UBE3B and UBE3A.

According to some embodiments of the invention, the substrate is selected from the group consisting of IKZF1, IKZF2, IKZF3, CK1 alpha, c-myc, k-ras, PHD3, SPROUTY2, Mitofusin 1, 2, MIRO, NEMO, SMADs, TβR-I, P53, SSA, HHR23, EPHEXIN5, ARC, PPARα, cyclin-B, Cdc25C and Calmodulin.

According to some embodiments of the invention, the agent is not ubiquitin. According to some embodiments of the invention, the agent is a small molecule agent.

According to some embodiments of the invention, the bacterial cell further expresses an E1 activating enzyme and/or an E2 conjugating enzyme.

According to some embodiments of the invention, the selectable signal is a selectable polypeptide.

According to some embodiments of the invention, the selectable polypeptide is a split antibiotic resistance polypeptide.

According to some embodiments of the invention, the antibiotic resistance polypeptide is selected from the group consisting of chloramphenicol, DHFR or Beta lactamase.

According to some embodiments of the invention, the first polypeptide is attached to the ubiquitin via a linker.

According to some embodiments of the invention, the detectable signal is a detectable polypeptide.

According to some embodiments of the invention, the detectable polypeptide is an optically detectable polypeptide.

According to some embodiments of the invention, the detectable polypeptide is selected from the group consisting of a split fluorescent polypeptide, a split luminescent polypeptide and a split phosphorescent polypeptide.

According to some embodiments of the invention, the ubiquitinating enzyme is a ubiquitin E3-ligase.

According to some embodiments of the invention, the ubiquitin E3-ligase is selected from the group consisting of CRBN, betaTrcP, VHL-ElonginB-ElonginC-Hif1 complex, Siah2, Smurf1, MDM2, BRCA1, PARKIN, UBE3A, TRIM5, NEDD4, UBR5, ITCH and Huwe1.

According to some embodiments of the invention, the substrate is selected from the group consisting of IKZF1, IKZF2, IKZF3, CK1 alpha, c-myc, k-ras PHD3, SPROUTY2, Mitofusin 1, 2, MIRO, NEMO, SMADs, TβR-I, P53, SSA, HHR23, EPHEXIN5, ARC, PPARα, cyclin-B, Cdc25C and Calmodulin.

According to some embodiments of the invention, the second polypeptide is not a ubiquitinating enzyme.

According to some embodiments of the invention, the agent is not ubiquitin.

According to some embodiments of the invention, the agent is a small molecule agent.

According to some embodiments of the invention, the ubiquitinating enzyme further comprises an E1 activating enzyme and an E2 conjugating enzyme.

According to some embodiments of the invention, the selectable signal is a selectable polypeptide.

According to some embodiments of the invention, the selectable polypeptide is a split antibiotic resistance polypeptide.

According to some embodiments of the invention, the antibiotic resistance polypeptide is selected from the group consisting of chloramphenicol, DHFR or Beta lactamase.

According to some embodiments of the invention, the second polypeptide is attached to the ubiquitin via a linker.

According to some embodiments of the invention, the detectable signal is a detectable polypeptide.

According to some embodiments of the invention, the detectable polypeptide is an optically detectable polypeptide.

According to some embodiments of the invention, the detectable polypeptide is selected from the group consisting of a split fluorescent polypeptide, a split luminescent polypeptide and a split phosphorescent polypeptide.

According to some embodiments of the invention, the ubiquitin E3-ligase is selected from the group consisting of CRBN, betaTrcP, VHL-ElonginB-ElonginC-Hif1 complex, Siah2, Smurf1, MDM2, BRCA1, PARKIN, UBE3A, TRIM5, NEDD4, UBR5, ITCH and Huwe1.

According to some embodiments of the invention, the ubiquitin E3-ligase is a chimeric ubiquitin E3-ligase.

According to some embodiments of the invention, the test polypeptide is selected from the group consisting of IKZF1, IKZF2, IKZF3, CK1 alpha, c-myc, k-ras, PHD3, SPROUTY2, Mitofusin 1, 2, MIRO, NEMO, SMADs, TβR-I, P53, SSA, HHR23, EPHEXIN5, ARC, PPARα, cyclin-B, Cdc25C and Calmodulin.

According to some embodiments of the invention, the bacterial cell further expresses an E1 activating enzyme and an E2 conjugating enzyme.

According to some embodiments of the invention, the bacterial cell expresses:

(a) ubiquitin attached to a first polypeptide fragment; and

(b) the test polypeptide attached to a second polypeptide fragment, wherein the first polypeptide fragment associates with the second polypeptide fragment to generate a reporter polypeptide on ubiquitination of the substrate.

According to some embodiments of the invention, the reporter polypeptide comprises a selectable polypeptide.

According to some embodiments of the invention, the selectable polypeptide is a split antibiotic resistance polypeptide.

According to some embodiments of the invention, the split antibiotic resistance polypeptide is chloramphenicol, DHFR or B lactamase.

According to some embodiments of the invention, the first polypeptide fragment is attached to the ubiquitin via a linker.

According to some embodiments of the invention, the second polypeptide fragment is attached to the test polypeptide via a linker.

According to some embodiments of the invention, the reporter polypeptide is an optically detectable signal.

According to some embodiments of the invention, the optically detectable polypeptide is selected from the group consisting of a split fluorescent polypeptide, a split luminescent polypeptide and a split phosphorescent polypeptide.

According to some embodiments of the invention, the agent is a small molecule agent.

According to some embodiments of the invention, the agent comprises a glutarimide ring.

According to some embodiments of the invention, the agent is a thalidomide.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a scheme showing how one can identify small molecules (e.g. glutarimide like molecules) that mediate the binding of a first polypeptide (e.g. A) to a second polypeptide (e.g. B). The affinity of the molecule to each of the proteins may be very weak. However, due to ubiquitination, the transient weak interaction is translated into a stable covalent bond—ubiquitination.

FIG. 2 is a scheme showing how one can identify small molecules (e.g. glutarimide like molecules) that mediate the binding of a first polypeptide (e.g. A) to a second polypeptide (e.g. B). In this scheme the screen is for only half the molecule, where the full black pentagon is known to bind protein A and the screen is for covalently attached molecules to this pentagon which bind to protein B.

FIG. 3 is a scheme showing how one can identify small molecules (e.g. glutarimide like molecules) that mediate the binding of an E3 ligase to a second protein of interest (POI).

FIGS. 4A-B are schemes which show exemplary systems for identifying small molecules that mediate the binding of a CRL E3 ligase and target protein.

FIG. 5 is a scheme which shows an exemplary system for identifying small molecules that mediate the binding of a CRL-CRBN E3 ligase and target protein.

FIGS. 6A-B provides the sequence of CAT. The DNA sequence and the translation products of CAT′ is illustrated in FIG. 6A (SEQ ID NO: 1 for the protein and SEQ ID NO: 8 for the DNA sequence). Arrow marks the cleavage site that was chosen for the split protein fragments (A). The DNA sequence and the translation products of the split-CAT′ fragments are shown in FIG. 6B. The N terminal protein sequence is as set forth in SEQ ID NO: 2. The N terminal DNA sequence is as set forth in SEQ ID NO: 4. The C terminal protein sequence is as set forth in SEQ ID NO: 3. The C terminal DNA sequence is as set forth in SEQ ID NO: 5. The stop codon after residue Q30 and the initiation codon prior residue C31 are shown in the split protein fragments (B).

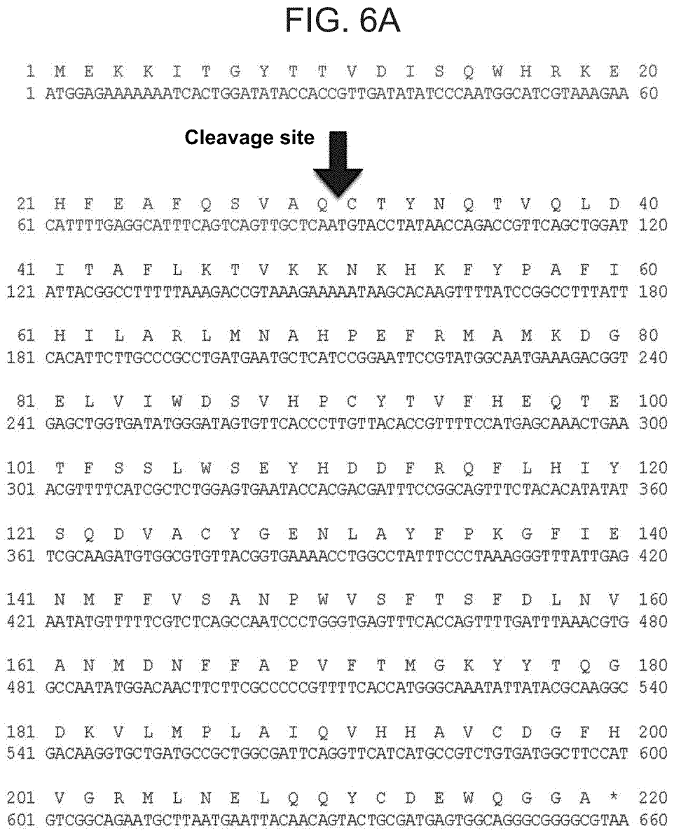

FIG. 7 provides a list of CAT proteins that have a homology range of 79-100% identity to SEQ ID NO: 1. Arrows represent the conserved split site among all the family protein members. Accession codes are in blue. The sequence AFQSVAQCTYNQTVQLDI is set forth in SEQ ID NO: 11.

FIG. 8 is a scheme which shows an exemplary system for identifying small molecules that mediate the binding of a CRL-CRBN E3 ligase and target protein. In this scheme the CRBN E3 ligase is fused in frame with its corresponding E2.

FIG. 9 is a scheme which shows an exemplary system for corroborating that Thalidomide and Lenalidomide (TLD/LLD) significantly promote the growth of E. coli that express IKZF1 and the CRBN ubiquitylation cascade.

FIGS. 10A-C are graphs illustrating the results of PROTAC experiment with split-CAT in rich LB media. Shown Thalidomide (TLD; FIG. 9A) and Lenalidomide (LLD, FIG. 9B) growth dependent of IKZF1 ubiquitylation. GSPT1 (FIG. 9C) serves as negative control as its ubiquitylation is not promoted by TLD.

FIGS. 11A-C are graphs illustrating the results of E. coli expressing ubiquitylation selection system including human Ub, wheat E1, yeast Ubc4 (E2), human CRBN (E3) and human IKZF as ubiquitylation target. Growth was monitored by measuring the O.D. 600 nm. Thalidomide (TLD) or Lenalidomide (LLD) were added at time zero with the indicated concentrations (A, B respectively). A classical bell-shape PROTAC dependent ubiquitylation (as measured by growth) is shown in C.

FIGS. 12A-B are graphs illustrating thalidomide/lenalidomide dependent ubiquitylation of CK1α. A. A selective growth assay of E. coli cells in the presence of chloramphenicol. The cells were transformed with the split-CAT system that expresses CK1α as ubiquitylation target. B. Classical bell-shape response curve for increased LLD concentrations.

FIG. 13 is a graph illustrating CC-885 dependent ubiquitylation of GSPT1 by CRBN. A classical bell-shaped activity (growth) curve is demonstrated for GSPT1 ubiquitylation by CRBN pending increased concentration of CC-885. E. coli were transformed with GSPT1 and Ub tethered to the split-CAT selection system and were co-expressed with CRBN ligase yeast Ubc4 and Wheat E1. Growth under selective conditions (with chloramphenicol) and increased concentration of the CC-885 PROTAC molecule is shown.

FIG. 14 is a scheme which shows an exemplary system for identifying small molecules that mediate the binding of an E3 ligase to an E2 conjugating enzyme.

### DESCRIPTION OF SPECIFIC EMBODIMENTS OF THE INVENTION

The present invention, in some embodiments thereof, relates to bacterial systems for screening small molecules of interest.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

About half of the eukaryotic proteome undergoes ubiquitination, but most of the enzymatic cascades leading to substrate modification are still unknown. The present inventors have invented a genetic selection tool that utilizes E. coli, which lack deubiquitylases and ubiquitin dependent degradation, to identify interactions along ubiquitination cascades. Co-expression of split antibiotic resistance protein tethered to ubiquitin and ubiquitination target together with a functional ubiquitination apparatus results in a covalent assembly of the resistance protein, giving rise to bacterial growth on selective media. The capability of the screening tool for small molecule modulators, in a high-throughput format is demonstrated herein.

Thus, according to one aspect of the present invention there is provided a method of identifying an agent which mediates the binding of a first polypeptide to a second polypeptide comprising:

(a) contacting a bacterial cell which expresses the first polypeptide and the second polypeptide with the agent, wherein the bacterial cell outputs a detectable or selectable signal which correlates with the non-direct binding of the first polypeptide to the second polypeptide via the agent, wherein the non-direct binding of the first polypeptide to the second polypeptide via the agent brings about the ubiquitination of the first polypeptide or the second polypeptide; and

(b) measuring the level or the rate of accumulation of the detectable or selectable signal, wherein a change in the level as compared to the level in the absence of the agent, is indicative of an agent which mediates the binding of the first polypeptide to the second polypeptide.

The method of this aspect of the present invention can be used to screen for agents that mediate the binding of a first polypeptide to a second polypeptide i.e. that are capable of binding to both the first and second polypeptide concurrently, serving as a bridge between the two polypeptides. In one embodiment, the first polypeptide is capable of binding very weakly (e.g. with a Kd above 200 μM) to the second polypeptide in the absence of the mediating agent. The mediating agent thus augments the binding of the first polypeptide to the second polypeptide (e.g. increases the binding by at least 10 fold). In another embodiment, the first polypeptide is only capable of binding the second polypeptide in the presence of the mediating agent.

The agents of this aspect of the present invention are typically heterobifunctional, comprising a ligand which binds to a first target protein, a ligand which binds to a second target protein (e.g. an E3 ubiquitin ligase) and optionally a linker connecting the two ligands. The ligand which binds to the first and second target protein are typically not identical.

It will be appreciated that the agent of this aspect of the present invention is not ubiquitin.

According to a particular embodiment, the mediating agent is a chimeric agent.

Agents that may be screened include small molecule agents, e.g. small molecule agents which comprise a glutarimide ring, thalidomide and thalidomide like molecules such as lenalidomide, pomalidomide, CC-885, peptide agents, nucleic acid agents, antibodies, proteins, chemotherapeutic agents etc.

The screening assay of this aspect of the present invention uses bacteria that have been genetically modified to output a detectable or selectable signal which correlates with the ability of the substrate to mediate the binding between the two polypeptides. The system further stabilizes this binding by introducing a stable covalent bond (ubiquitination) between the two proteins.

Ubiquitination takes place by a cascade of enzyme activity (i.e. a plurality of enzymes which work together to bring about the same function—ubiquitination). For example, E1 activates the Ub; then Ub is transferred to E2. E2 together with E3 (or in many cases transfer the Ub to E3) recognize a specific target and ligate the Ub to the target protein.

Below is a list of some exemplary components of the assay which may be expressed by the genetically modified bacteria of this aspect of the present invention, each of which will be described in detail herein below.

1. Ubiquitin;

2. Detectable signal;

3. At least one ubiquitinating enzyme; and

4. Substrate (target for ubiquitylation).

Any bacteria can be used for this assay so long as it lacks endogenous significant deubiquitinase activity and preferably also endogenous ubitquitinase activity. In one embodiment, the bacteria has at least 10 fold less endogenous deubiquitinase activity and endogenous ubiquitinase activity than a human cell. In another embodiment, the bacteria has at least 20 fold less endogenous deubiquitinase activity and endogenous ubiquitinase activity than a human cell.

Preferably the bacteria lack resistance to the selection markers in the current system. Examples of such bacteria include, but are not limited to E. coli K-12 derivatives including W3110, MG1655, DH5a, JM101, JM19, BL21, B834, XL1-Blue; also other non E. coli bacteria may be used.

According to a particular embodiment, the bacteria used in the system are of the genus Escherichia, such as for example E. coli.

In order to express the components of the assay, a polynucleotide sequence encoding the elements described above is preferably ligated into a nucleic acid construct suitable for bacterial cell expression. Such a nucleic acid construct includes a promoter sequence for directing transcription of the polynucleotide sequence in the cell in a constitutive or inducible manner.

The phrase “an isolated polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein, the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase; or synthetically synthesized by assembled from short oligonucleotide.

As used herein, the phrase “genomic polynucleotide sequence” refers to a sequence derived (isolated) from a chromosome and thus it represents a contiguous portion of a chromosome.

As used herein, the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Exemplary promoters contemplated by the present invention include, but are not limited to polyoma, Simian Virus 40 (SV40), adenovirus, retroviruses, hepatitis-B virus and cytomegalovirus promoters. According to a particular embodiment, the promoter is a bacterial promoter.

Constitutive promoters suitable for use with the present invention are promoter sequences which are active under most environmental conditions and most types of bacterial cells such as an unregulated bacteriophage lambda left promoter (pL) or pTac which presents high leakiness.

Enhancer elements can stimulate transcription up to 1,000 fold from linked homologous or heterologous promoters. Enhancers are active when placed downstream or upstream from the transcription initiation site. Many enhancing elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

In a preferred embodiment, the vector comprises a bacterial replication of origin.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

The use of bacterial operon architecture for multi-gene expression, where a single promoter is followed by several open reading frames (ORFs) each contains a ribosome-binding site (Shine-Dalgarno sequence) facilitates the co-expression of the multi-protein complex of the ubiquitination apparatus.

It will be appreciated that the individual elements comprised in the expression vector can be arranged in a variety of configurations. For example, enhancer elements, promoters and the like, and even the polynucleotide sequence(s) encoding the fusion protein can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Exemplary methods of introducing the polynucleotides of the present invention into prokaryotic cells are well known in the art—these include, but are not limited to, transforming with a recombinant bacteriophage DNA, plasmid DNA or cosmid DNA expression vector containing the relevant gene sequences.

Other than containing the necessary elements for the transcription and translation of the inserted coding sequence, the expression construct of some embodiments of the invention can also include sequences engineered to enhance stability, production or isolation of the expressed peptide fragments.

Examples of bacterial constructs include the pET series of E. Coli expression vectors (see for example Studier et al (1990) Methods in Enzymol 185:60-89) in which their T7 promoter was replaced with the constitutive active bacteriophage pH (left promoter). Other vectors that may be used are those that belong to the pZE vector family (e.g. pZE21), those that belong to the pCloDF (containing a pCloDF13 origin) and pACYC (containing a p15A origin of replication).

Ubiquitin

The term “ubiquitin” as used herein refers to either mammalian ubiquitin having a sequence as set forth in SEQ ID NO: 9 or yeast ubiquitin having a sequence as set forth in SEQ ID NO: 10.

Detectable Signal

In one example, the detectable signal is a fluorescent protein or an enzyme producing a colorimetric reaction. Exemplary proteins that generate a detectable signal include, but are not limited to green fluorescent protein (Genbank Accession No. AAL33912), alkaline phosphatase (Genbank Accession No. AAK73766), peroxidase (Genbank Accession No. NP_568674), histidine tag (Genbank Accession No. AAK09208), Myc tag (Genbank Accession No. AF329457), biotin ligase tag (Genbank Accession No. NP_561589), orange fluorescent protein (Genbank Accession No. AAL33917), beta galactosidase (Genbank Accession No. NM_125776), Fluorescein isothiocyanate (Genbank Accession No. AAF22695) and strepavidin (Genbank Accession No. S11540).

In another example, the detectable signal is a luminescent protein such as products of bacterial luciferase genes, e.g., the luciferase genes encoded by Vibrio harveyi, Vibrio fischeri, and Xenorhabdus luminescens, the firefly luciferase gene FFlux, and the like.

In one embodiment, the selection is dominant selection, which typically uses a drug to arrest growth of a host cell. Those cells which would express a protein conveying drug resistance would survive the selection. The use of split marker allows the detection of ubiquitination events as further described below.

In order to output a detectable or selectable signal which correlates with the binding of the first polypeptide to the second polypeptide, the present inventors contemplate using a split protein. When the protein is expressed as two “split” fragments, there is no detectable or selectable signal. However, when the two fragments are brought close enough together (i.e. on binding of the first and second polypeptide via the agent and subsequent ubiquitination of at least one of the polypeptides) they form a functional protein that emits a detectable or selectable signal—i.e. generate a reporter protein.

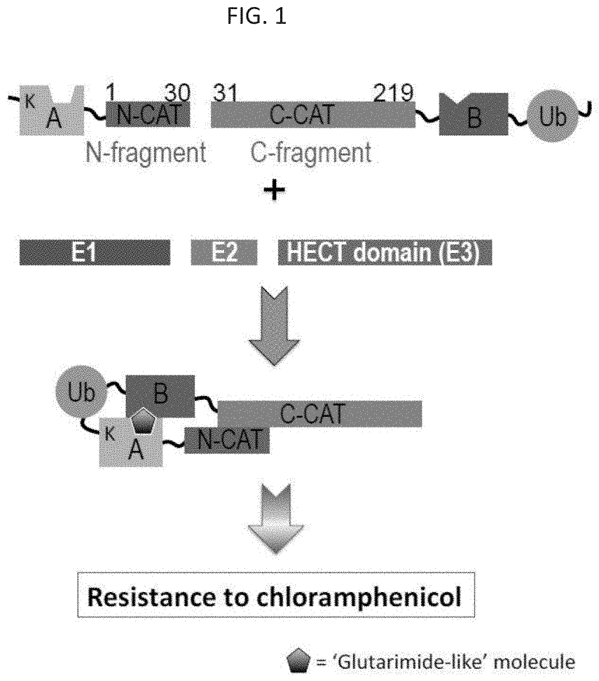

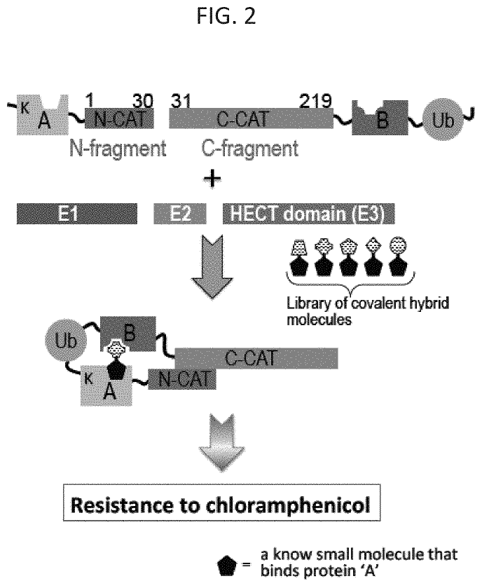

In one embodiment, one fragment of the split protein is linked to ubiquitin via the first polypeptide and the other fragment of the split protein is linked to the second polypeptide—see for example FIGS. 1 and 2. In another embodiment, one fragment of the split protein is linked to the first polypeptide via ubiquitin and the other fragment of the split protein is linked to the second polypeptide. In either of these scenarios, the second polypeptide serves as a substrate for ubiquitination.

In the presence of the correct agent (i.e. one which concurrently binds both the first and second polypeptides), the first polypeptide becomes indirectly bound to the second polypeptide. The two polypeptides are brought close enough together such that in the presence of a suitable ubiquitinating enzyme/enzymes, the ubiquitin which is already bound to the first polypeptide also binds to the second polypeptide. In this way the two fragments of the split protein are brought close enough together and are bound sufficiently tightly to allow for a detectable or selectable signal.

In another embodiment, one fragment of the split protein is linked to ubiquitin and the other fragment of the split protein is linked to a first polypeptide (e.g. β-Catenin). In this scenario, the first polypeptide serves as a substrate for ubiquitination.

The second polypeptide is a ubiquitinating enzyme—e.g. E3 ubiquitin ligase. An exemplary E3 ligase is the E3 ligase cereblon (CRBN; e.g. Q96SW2) or the homologous to the E6-AP carboxyl terminus (HECT) domains. In the case of β-Catenin, the E3 enzyme SCF^{β-TrCP} is contemplated.

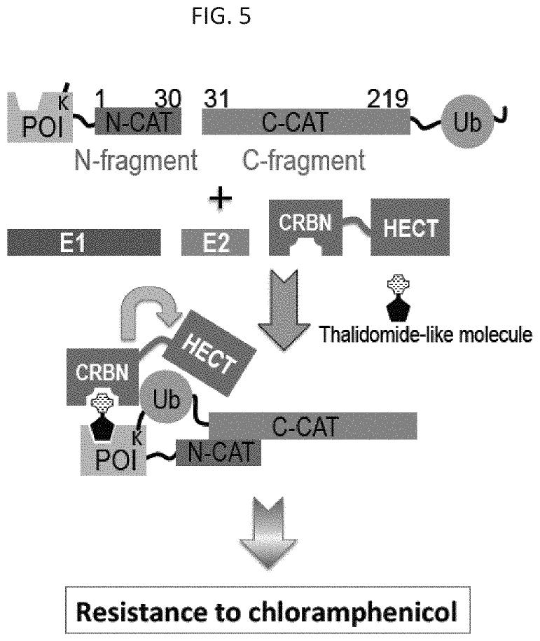

Optionally, the E3 ligase is a chimeric E3 ligase comprising two different ligases. The E3 ligase for which the PROTAC is being sought may be genetically modified such that it no longer comprises E3 ligase activity. The modified E3 ligase can then be expressed as a fusion protein with a polypeptide that comprises E3 ligase activity—e.g. a modified CRBN (devoid of E3 ligase activity) which is attached to a HECT domain (see for example FIG. 5).

Alternatively, the E3 ligase is a chimeric E3 ligase, comprising both an E3 ligase and E2 (Ubiquitin conjugating enzyme—e.g. UBC4 yeast Ubiquitin-conjugating enzyme—P15731). The E3/E2 protein can be expressed as a fusion protein whereby the E3 is in-frame with the E2.

In the presence of the correct agent (i.e. one which binds both the first polypeptide and the E3 ligase), the first polypeptide becomes indirectly bound to the E3 ligase. The E3 ligase and the polypeptide are brought close enough together such that the ubiquitin which is already bound to the first fragment of the split protein also binds to the first polypeptide. In this way, the two fragments of the split protein are brought close enough together and are bound sufficiently tightly to allow for a detectable or selectable signal.

According to a particular embodiment, the split protein combines to generate a reporter protein which is fluorescent, luminescent, phosphorescent or one that confers antibiotic resistance.

Examples of split proteins contemplated by the present invention include, but are not limited to beta lactamase, dihydrofolate reductase (DHFR), focal adhesion kinase, enhanced GFP, horseradish peroxidase, Infrared fluorescent protein IFP1.4 (an engineered chromophore-binding domain (CBD) of a bacteriophytochrome from Deinococcus radiodurans) LacZ (beta-galactosidase)’ Luciferase, TEV (Tobacco etch virus protease).

According to a particular embodiment the split protein provides resistance to an antibiotic when combined, but the bacteria is susceptible to the antibiotic when split. Preferably, the split protein provides resistance to a bacteriostatic antibiotic when combined. Examples of bacteriostatic antibiotics include but are not limited to trimethoprim and chloramphenicol.

In the case of trimethoprim, a split DHFR protein may be expressed. Specifically the use of selective media which lack thymidine, glycine, serine or methionine and contains the trimethoprim antibiotic allows the selection of genes required for the ubiquitination process.

In the case of chloramphenicol, a split chloramphenicol acetyl transferase (CAT) enzyme can be expressed.

As used herein, the term CAT refers to an enzyme (EC 2.3.1.28) that catalyzes the acetyl-S-CoA-dependent acetylation of chloramphenicol at the 3-hydroxyl group.

An exemplary CAT construct system is described in PCT Application No. WO2019/030759, and may comprise:

(i) a first nucleic acid construct comprising a first polynucleotide having a nucleic acid sequence that encodes an N-terminal fragment of chloramphenicol acetyl transferase (CAT), the N-terminal fragment comprising a first portion of the catalytic active site of the CAT, the N-terminal fragment being devoid of acetylating activity; and

(ii) a second nucleic acid construct comprising a second polynucleotide having a nucleic acid sequence that encodes a C terminal fragment of the CAT, the C-terminal fragment comprising a second portion of the catalytic active site of the CAT, the C-terminal fragment being devoid of acetylating activity; and wherein the N-terminal fragment is capable of associating with the C-terminal fragment to generate an active CAT that is capable of acetylating chloramphenicol.

The construct system of the present invention is useful in detecting interaction between, for example, a known first member of a putative binding pair and a second member, for example one which was previously not known to bind the first member. The method detects the interaction of the first member with the second member by bringing into close proximity members of a fragment pair of the CAT reporter protein, such that the CAT reporter protein is reassembled to its original functionality or enzymatic activity. The fragments of the reporter protein of the present invention interact to bring about antibiotic resistance. This system enables, for example, the identification of molecules and/or genes that promote or inhibit key protein interactions, existing in a range of cell types, phyla and species, via high-throughput screens.

As used herein, the term CAT refers to an enzyme (EC 2.3.1.28) that catalyzes the acetyl-S-CoA-dependent acetylation of chloramphenicol at the 3-hydroxyl group.

In one embodiment, the CAT is CAT_{I}. In another embodiment, the CAT is CAT_{II}. In still another embodiment, the CAT is CAT_{III}.

The CAT of this embodiment may have an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% homologous or identical to the sequence as set forth in SEQ ID NO: 1, as determined using the Standard protein-protein BLAST [blastp] software of the NCBI.

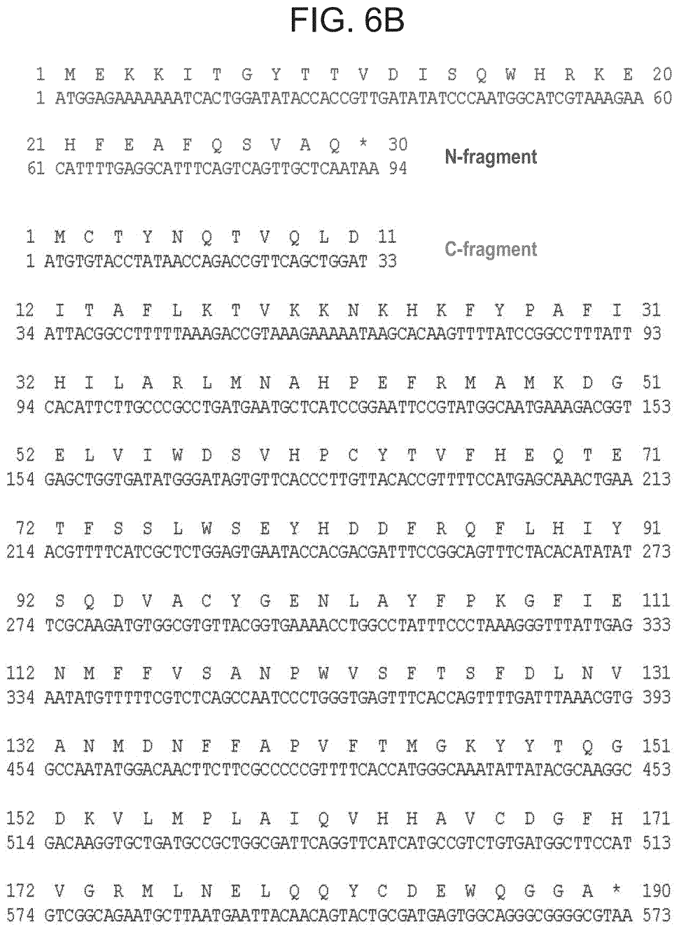

In one embodiment the CAT is an orthologue of CAT which comprises the amino acid sequence QC in its active site. Preferably the CAT orthologue comprises the sequence as set forth in SEQ ID NO: 11—such as those listed in FIG. 7.

In one embodiment the N-terminal fragment comprises a first portion of the catalytic active site of the CAT—e.g. the N terminal fragment typically contains the first 28 or 30 amino acids of the native CAT. The C-terminal fragment comprises the second portion of the catalytic active site of the CAT—for example, the C terminal fragment typically contains the rest of the sequence of the native CAT. The N-terminal fragment associates with the C-terminal fragment to generate an active CAT that is capable of acetylating chloramphenicol.

Preferably, the first amino acid of the C-terminal fragment is a small amino acid residue—for example cysteine or alanine. Thus, the C terminal fragment may begin with cysteine 31 (wherein the numbering is according to SEQ ID NO: 1), or alanine 29 (wherein the numbering is according to SEQ ID NO: 1). Other small amino acid residues include glycine, alanine, serine, proline, threonine, aspartate and asparagine.

By being small, the first amino acid of the C-terminal fragment after the formyl-methionine causes the latter to be post-translationally removed from the N-terminus (of the C-terminus fragment) hence salvaging the active site arrangement, as confirmed by the activity of the CAT.

In one embodiment, the N-terminal fragment comprises an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% homologous or identical to the sequence as set forth in SEQ ID NO: 2 or 6. The N-terminal fragment may consist of the sequence as set forth in SEQ ID NO: 2 or SEQ ID NO: 6.

The C-terminal fragment comprises an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% homologous or identical to the sequence as set forth in SEQ ID NO: 3 or 7.

The C-terminal fragment may consist of the sequence as set forth in SEQ ID NO: 3 or 7.

According to a particular embodiment, the N-terminal fragment is encoded by the nucleic acid sequence as set forth in SEQ ID NO: 4.

The C-terminal fragment may be encoded by the nucleic acid sequence as set forth in SEQ ID NO: 5.

In any of the above described embodiments, the linking of ubiquitin (also referred to herein as the first component) to either the polypeptides or the split protein fragments (also referred to herein as the second component) may be direct or via a linker—i.e. a linking moiety.

Furthermore, the linking of the split protein fragment to either of the polypeptides may be direct or via a linking moiety. (In this case, the split protein fragment may be referred to as the first component and the linking moiety may be referred to as the second component).

Examples of linking moieties include but are not limited to a simple covalent bond, a flexible peptide linker, a disulfide bridge or a polymer such as polyethylene glycol (PEG). Peptide linkers may be entirely artificial (e.g., comprising 2 to 20 amino acid residues independently selected from the group consisting of glycine, serine, asparagine, threonine and alanine) or adopted from naturally occurring proteins. Disulfide bridge formation can be achieved, e.g., by addition of cysteine residues, as further described herein below. Linking through polyethylene glycols (PEG) can be achieved by reaction of the components having free cysteines with multifunctional PEGs, such as linear bis-maleimide PEGs. Alternatively, linking can be performed though the glycans on the component after their oxidation to aldehyde form and using multifunctional PEGs containing aldehyde-reactive groups.

Selection of the position of the link between the two components should take into account that the link should not substantially interfere with the ability of the ubiquitin to bind its target and for the first polypeptide to bind to the second polypeptide.

Thus, for example, the linking moiety is optionally a moiety which is covalently attached to a side chain, an N-terminus or a C-terminus of the first component, as well as to a side chain, an N-terminus or a C-terminus of the second component.

As mentioned, the linking moiety used in this aspect of the present invention may be a cysteine residue.

Thus, in some embodiments of the invention, each of the first and second components comprise at least one cysteine residue, such that the components are covalently linked to one another via a disulfide bridge formed between a cysteine residue in the first component and a cysteine residue in the second component.

Hereinthroughout, the phrases “disulfide bridge” and “disulfide bond” are used interchangeably, and describe a —S—S— bond.

The linker may comprise additional amino acids linked together by peptide bonds which serve as spacers such that the linker does not interfere with the biological activity of the final compound. The linker is preferably made up of amino acids linked together by peptide bonds. Thus, in preferred embodiments, the linker is made up of from 1 to 10 amino acids linked by peptide bonds, wherein the amino acids are selected from the 20 naturally occurring amino acids. Some of these amino acids may be glycosylated, as is well understood by those in the art. In a more preferred embodiment, besides cysteine the amino acids in the linker are selected from glycine, alanine, proline, asparagine, glutamine, and lysine. Even more preferably, besides cysteine, the linker is made up of a majority of amino acids that are sterically unhindered, such as glycine and alanine.

Thus, according to one embodiment the linker comprises the sequence cysteine-glycine.

Non-peptide linkers are also possible. For example, alkyl linkers such as —NH—(CH.sub.2).subs-C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker.

Thus, in some embodiments, at least one of the components is PEGylated or chemically modified to another form. PEGylation of the molecules can be carried out, e.g., according to the methods described in Youngster et al., Curr Pharm Des (2002), 8:2139; Grace et al., J Interferon Cytokine Res (2001), 21: 1103; Pepinsky et al., J Pharmacol Exp Ther (2001), 297:1059; Pettit et al., J Biol Chem (1997), 272:2312; Goodson et al. Biotechnology NY (1990), 8:343; Katre; J Immunol (1990), 144:209, Behrens et al US2006/0198819 A1, Klausen et al US2005/0113565 A1.

Preferably, the polyethylene glycol of the linker of the present invention is PEG 1000, 2000, 3000, 5000, 10000, 15000, 20000 or 40000 with PEG 20000 or 40000 being particularly preferred.

According to another embodiment the link is effected using a coupling agent.

The term “coupling agent”, as used herein, refers to a reagent that can catalyze or form a bond between two or more functional groups intra-molecularly, inter-molecularly or both. Coupling agents are widely used to increase polymeric networks and promote crosslinking between polymeric chains, hence, in the context of some embodiments of the present invention, the coupling agent is such that can promote crosslinking between polymeric chains; or such that can promote crosslinking between amino functional groups and carboxylic functional groups, or between other chemically compatible functional groups of polymeric chains. In some embodiments of the present invention the term “coupling agent” may be replaced with the term “crosslinking agent”. In some embodiments, one of the polymers serves as the coupling agent and acts as a crosslinking polymer.

By “chemically compatible” it is meant that two or more types of functional groups can react with one another so as to form a bond.

Exemplary functional groups which are typically present in gelatins and alginates include, but are not limited to, amines (mostly primary amines —NH₂), carboxyls (—CO₂H), sulfhydryls and hydroxyls (—SH and —OH respectively), and carbonyls (—COH aldehydes and —CO— ketones).

Primary amines occur at the N-terminus of polypeptide chains (called the alpha-amine), at the side chain of lysine (Lys, K) residues (the epsilon-amine), as found in gelatin, as well as in various naturally occurring polysaccharides and aminoglycosides. Because of its positive charge at physiologic conditions, primary amines are usually outward-facing (i.e., found on the outer surface) of proteins and other macromolecules; thus, they are usually accessible for conjugation.

Carboxyls occur at the C-terminus of polypeptide chain, at the side chains of aspartic acid (Asp, D) and glutamic acid (Glu, E), as well as in naturally occurring aminoglycosides and polysaccharides such as alginate. Like primary amines, carboxyls are usually on the surface of large polymeric compounds such as proteins and polysaccharides.

Sulfhydryls and hydroxyls occur in the side chain of cysteine (Cys, C) and serine, (Ser, S) respectively. Hydroxyls are abundant in polysaccharides and aminoglycosides.

Carbonyls as ketones or aldehydes can be form in glycoproteins, glycosides and polysaccharides by various oxidizing processes, synthetic and/or natural.

According to some embodiments of the present invention, the coupling agent can be selected according to the type of functional groups and the nature of the crosslinking bond that can be formed therebetween. For example, carboxyl coupling directly to an amine can be afforded using a carbodiimide type coupling agent, such as EDC; amines may be coupled to carboxyls, carbonyls and other reactive functional groups by N-hydroxysuccinimide esters (NHS-esters), imidoester, PFP-ester or hydroxymethyl phosphine; sulfhydryls may be coupled to carboxyls, carbonyls, amines and other reactive functional groups by maleimide, haloacetyl (bromo- or iodo-), pyridyldisulfide and vinyl sulfone; aldehydes as in oxidized carbohydrates, may be coupled to other reactive functional groups with hydrazide; and hydroxyl may be coupled to carboxyls, carbonyls, amines and other reactive functional groups with isocyanate.

Hence, suitable coupling agents that can be used in some embodiments of the present invention include, but are not limited to, carbodiimides, NHS-esters, imidoesters, PFP-esters or hydroxymethyl phosphines.

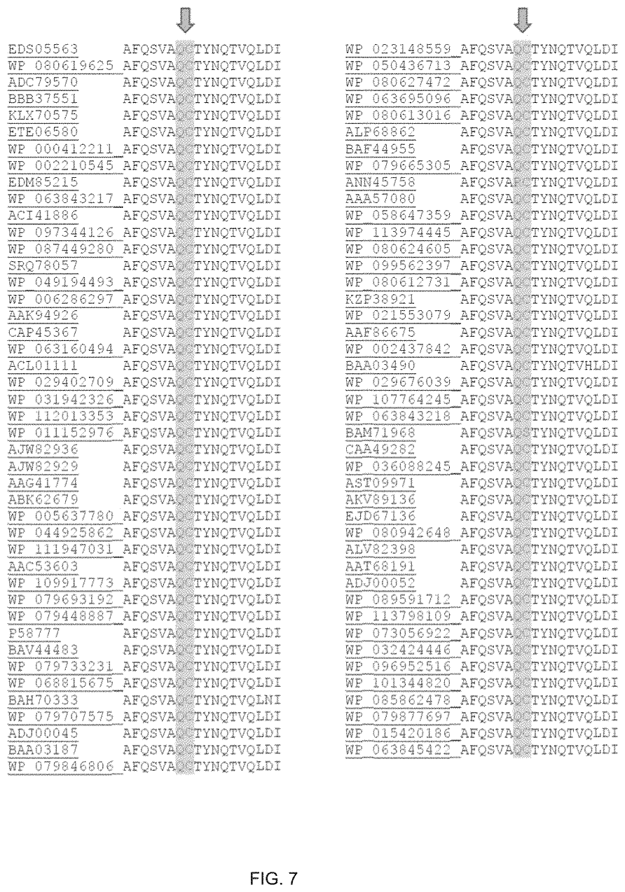

Exemplary systems that can be used to screen for small molecules are shown in FIGS. 1-5 and FIGS. 8 and 9.

Ubiquitinating Enzyme

As used herein, the term “ubiquitinating enzyme” refers to ubiquitin-activating enzymes (E1s), ubiquitin-conjugating enzymes (E2s) or ubiquitin ligases (E3s). Collectively they have the EC number EC 6.3.2.19.

In one embodiment, the ubiquitinating enzyme is a human ubiquitinating enzyme.

Ubiquitin-activating enzymes (E1s) have the EC number EC 6.2.1.45, ubiquitin-conjugating enzymes have the EC number EC 2.3.2.23 and ubiquitin ligases have the EC number 2.3.2.27.

Table 1, herein below provides nomenclature and most common synonyms used for E2 ubiquitin conjugating enzymes. The E2 nomenclature is in accordance with that used by the Human Genome Organization.

TABLE 1

| Human Genome Organization | |
|---|---|
| Nomenclature | Synonym |
| UBE2V2 | UEV2/MMS2 |
| UBE2D1 | UBC4/5/UBCH5A |
| UBE2D2 | UBC4/5/UBCH5B |
| UBE2D4 | HBUCE1 |
| UBE2D3 | UBC4/5 |
| UBE2W | FLJ11011 |
| UBE2B | UBC2/HHR6B/RAD6B/E217K |
| UBE2L6 | RIGB/UBCH8 |
| UBE2N | UBC13 |
| UBE2L3 | UBCH7 |
| UBE2G1 | UBC7/E217K |
| UBE2H | UBC8/E220K |
| UBE2M | UBC12 |
| UBE2F | NCE2 |
| UBE2E2 | UBCH8 |
| UBE2E3 | UBCH9/UBCM2 |
| UBE2S | E224K |
| UBE2U | MGC35130 |
| UBE2R1 | CDC34 |
| UBE2R2 | UBC3B/CDC34B |
| UBE2Z | HOYS7 |
| UBE2J2 | NCUBE2 |
| Probable ubiquitin- | LOC134111/FLJ25076 |
| conjugating enzyme | |
| E2 FLJ25076 | |
| AKTIP | FTS/FT1 |
| UBE2J1 | NCUBE1 |
| UBE2V1 | UEV1/CROC1 |
| UBE2Q2 | DKFZ/UBCI |
| UBE2Q1 | NICE5 |
| | TSG101/VPS23/SG10 |
| UEVLD | UEV3 |

In one embodiment, the ubiquitinating enzyme is an E3 ligase.

Exemplary E3 ligases contemplated by the present invention include, but are not limited to CRBN, HECT, betaTrcP, Siah2, Smurf1, MDM2, BRCA1, PARKIN, UBE3A, TRIM5, NEDD4, UBR5, Huwe1, Arkadia, ITCH, MuRF1, TRAF6, Trim32, UBR4, UBE3B and UBE3D.

Below is a brief description of exemplary E3 ligases contemplated by the present invention and some of their exemplary substrates.

Seven in Absentia Homolog 2 (SIAH2):

SIAH2 is a RING finger type ubiquitin ligases with a catalytic RING domain on its N-terminus, followed by two zinc fingers and a C-terminal substrate binding domain.

Siah2 is an E3 ubiquitin ligase implicated in diverse biological processes including p38/JNK/NF-kB signaling pathways, DNA damage, estrogen signaling, programmed cell death, Ras/Raf pathway, mitosis, and hypoxia.

Siah2 targets numerous substrates for degradation including TRAF2 (ketoglutarate dehydrogenase), Spry2 (Sprouty2), and the prolyl hydroxylase PHD3.

Siah2 also limits its own availability through self-ubiquitination and degradation.

Siah2 play a key role in hypoxia, through the regulation of HIF-1α transcription stability and activity via regulation of PHD3 stability.

Smad Ubiquitination Regulatory Factor-1 (Smurf1)

Smurf1 is a NEDD4-like Class IV HECT (homologous to E6-AP carboxylterminus) family E3 ligase with catalytic activity.

Smurf1 has been linked to several important biological pathways, including the bone morphogenetic protein pathway, the non-canonical Wnt pathway, and the mitogen-activated protein kinase pathway.

Smurfs possess three functional domains: an N-terminal protein kinase C (PKC)-related C2 domain which binds to phospholipids, targeting Smurfs to intracellular membranes, a central region containing two to four WW (tryptophan residues) protein—interacting domains which mediate ligase-substrate associations through interactions with a variety of proline-rich (PPXY) motifs and proline-containing phosphoserine/phosphothreonine sequences of the protein substrate, and a C-terminal HECT domain, responsible for ubiquitin transfer from a conserved cysteine residue at position 716 to a lysine residue in a substrate protein.

Smurf1 promotes p53 degradation by enhancing the activity of the E3 ligase MDM2. Smurf1 stabilizes MDM2 by enhancing the heterodimerization of MDM2 with MDMX, during which Smurf1 interacts with MDM2 and MDMX.

Smurf1 is also a key negative regulator of transforming growth factor (TGF)-β/bone morphogenetic protein (BMP) signaling pathway.

Mouse double minute 2 homolog (MDM2) MDM2 also known as E3 ubiquitin-protein ligase Mdm2 is an important negative regulator of the p53 tumor suppressor.

Mdm2 protein functions both as an E3 ubiquitin ligase that recognizes the N-terminal trans-activation domain (TAD) of the p53 tumor suppressor and as an inhibitor of p53 transcriptional activation.

Mdm2 contains a C-terminal RING domain (amino acid residues 430-480), which contains a Cis3-His2-Cis3 consensus that coordinates two molecules of zinc. These residues are required for zinc binding, which is essential for proper folding of the RING domain. The RING domain of Mdm2 confers E3 ubiquitin ligase activity and is sufficient for E3 ligase activity in Mdm2 RING autoubiquitination. The RING domain of Mdm2 is unique in that it incorporates a conserved Walker A or P-loop motif characteristic of nucleotide binding proteins, as well as a nucleolar localization sequence.

Mdm2 is capable of auto-polyubiquitination, and in complex with p300, a cooperating E3 ubiquitin ligase, is capable of polyubiquitinating p53. In this manner, Mdm2 and p53 are the members of a negative feedback control loop that keeps the level of p53 low in the absence of p53-stabilizing signals.

BRCA1

BRCA1-BARD1 constitutes a heterodimeric RING finger complex of the BRCA1/BRCA2-containing complex (BRCC) that contains significant ubiquitin ligase activity.

BRCA1 plays critical roles in the repair of chromosomal damage (error-free repair of DNA double-strand breaks), cell cycle checkpoint control, and genomic stability.

BRCA1 forms several distinct complexes through association with different adaptor proteins, and each complex forms in a mutually exclusive manner.

BRCA1 combines with other tumor suppressors, DNA damage sensors and signal transducers to form a large multi-subunit protein complex known as the BRCA1-associated genome surveillance complex (BASC).

The BRCA1 protein associates with RNA polymerase II, and through the C-terminal domain, also interacts with histone deacetylase complexes. Thus, this protein plays a role in transcription, DNA repair of double-strand breaks ubiquitination, transcriptional regulation as well as other functions

Parkin:

Parkin is a RING-between-RING E3 ligase that functions in the covalent attachment of ubiquitin to specific substrates.

It is best known for regulating the disposal of dysfunctional mitochondria (together with PINK1, a serine threonine kinase) via mitochondrial autophagy (i.e., mitophagy).

Upon loss of mitochondrial membrane potential, PINK1 becomes stabilized and activated on the outer mitochondrial membrane (OMM), resulting in recruitment and activation of Parkin.

Parkin facilitates ubiquitination of a broad number of targets expressed on the OMM (e.g., TOM20, Mitofusins, VDAC, Fis1) resulting in recruitment of the autophagy machinery, autophagosome formation and mitochondrial clearance.

In addition to its established role in mitophagy and UPS, Parkin impacts other neuroprotective cellular pathways, including TNFα signaling, and Wnt/β catenin signaling, and is also a putative tumor suppressor.

UBE3A (Gene Coding for E6-Associated Protein; E6-AP):

This ligase promotes the ubiquitylation and degradation of p53. E6-AP was subsequently shown to ubiquitylate proteins independent of E6 and to serve an independent secondary function as a transcriptional co-activator of nuclear estrogen receptors. E6-AP has been implicated in a broad range of processes (e.g. cell growth, synaptic formation and function, etc.) and has been shown to have many different target substrates (e.g., HHR23A, CDKN1B, MCMI, etc.).

Tripartite Motif-5 (TRIM5):

This ligase is a RING finger E3 ligase a key anti-viral restriction factor and directly involved in inhibiting HIV-1 replication.

Neural Precursor Cell Expressed Developmentally Downregulated 4 (NEDD4-1):

Nedd4-1 ubiquitinates a number of substrates, including ENaC, ADRB2, AMPA, Notch, pAKT, VEGFR2, EPS15, LATS1, and MDM2.

The vacuolar protein sorting protein Alix recruits NEDD4 to HIV-1 Gag protein to facilitate HIV-1 release via a mechanism that involves Alix ubiquitination.

NEDD4 also binds and ubiquitinates the latent membrane protein 2A (LMP2A) of the Epstein-Bar virus (EBV) to activate B-cell signal transduction.

Ubiquitin Protein Ligase E3 Component n-Recognin 5 (UBR5):

This ligase is also known as EDD (E3 identified by Differential Display), EDD1, HHYD, KIAA0896, or DDS.

URB5 acts as a general tumor suppressor by ubiquitinating, which increases p53 levels and induces cell senescence. UBR5 also ubiquitinates TopBP1, a topo-isomerase that intervenes in DNA damage response.

HUWE1:

HUWE1 (also known as ARF-BP1, MULE, LASU1, or HECTH9) is an E3 ligase that regulates the stability of diverse cellular substrates and, in consequence, numerous physiological processes, including DNA replication and damage repair, cell proliferation and differentiation, and apoptosis.

HUWE1 substrates include both tumor promoters (e.g., N-MYC, C-MYC, MCL1) and suppressors (e.g., p53, MYC, MIZ1).

HUWE1 has demonstrated both pro-oncogenic and tumor-suppressor functions in different tumor models.

HUWE1 belongs to the HECT (Homologous to E6AP C-Terminus)-family of ubiquitin E3 ligases.

Other additional E3 ligases and their substrates are provided in Table 2, herein below.

TABLE 2

| Ligase | Substrate | Function |
|---|---|---|
| AMFR | KAI1 | AMFR is also known as gp78. AMFR |
| | | is an integral ER membrane protein |
| | | and functions in ER-associated |
| | | degradation (ERAD). AMFR has |
| | | been found to promote tumor |
| | | metastasis through ubiquitination of |
| | | the metastasis supressor, KAI1. |
| APC/Cdc20 | Cyclin B | The anaphase promoting |
| | | complex/cyclosome (APC/C) is a |
| | | multiprotein complex with E3 ligase |
| | | activity that regulates cell cycle |
| | | progression through degradation of |
| | | cyclins and other mitotic proteins. |
| | | APC is found in a complex with |
| | | CDC20, CDC27, SPATC1, and |
| | | TUBG1. |
| APC/Cdh1 | Cdc20, | The anaphase promoting |
| | Cyclin B, | complex/cyclosome (APC/C) is a |
| | Cyclin A, | multiprotein complex with E3 ligase |
| | Aurora A, | activity that regulates cell cycle |
| | Securin, | progression through degradation of |
| | Skp2, | cyclins and other mitotic proteins. |
| | Claspin | The APC/C-Cdh1 dimeric complex is |
| | | activated during anaphase and |
| | | telophase, and remains active until |
| | | onset of the next S phase. |
| C6orf157 | Cyclin B | C6orf157 is also known as H10BH. |
| | | C6orf157 is an E3 ubiquitin ligase |
| | | that has been shown to ubiquitinate |
| | | cyclin B. |
| Cbl | | Cbl-b and c-Cbl are members of the |
| | | Cbl family of adaptor proteins that |
| | | are highly expressed in hematopoietic |
| | | cells. Cbl proteins possess E3 |
| | | ubiquitin ligase activity that |
| | | downregulates numerous signaling |
| | | proteins and RTKs in several |
| | | pathways such as EGFR, T cell and B |
| | | cell receptors, and integrin receptors. |
| | | Cbl proteins play an important role in |
| | | T cell receptor signaling pathways. |
| CBLL1 | CDH1 | CBLL1 is also known as Hakai. |
| | | CBLL1 is an E3 ubiquitin ligase that |
| | | ubiquitinates the phosphorylated form |
| | | of E-Cadherin, causing its |
| | | degradation and loss of cell-cell |
| | | adhesions. |
| CHFR | PLK1, | CHFR is an E3 ubiquitin ligase that |
| | Aurora A | functions as a mitotic stress |
| | | checkpoint protein that delays entry |
| | | into mitosis in response to stress. |
| | | CHFR has been shown to ubiquitinate |
| | | and degrade the kinases PLK1 and |
| | | Aurora A. |
| CHIP | HSP70/90, | CHIP is an E3 ubiquitin ligase that |
| | iNOS, | acts as a co-chaperone protein and |
| | Runx1, | interacts with several heat shock |
| | LRRK2 | proteins, including HSP70 and |
| | | HSP90, as well as the non-heat shock |
| | | proteins iNOS, Runx1 and LRRK2. |
| DTL (Cdt2) | p21 | DTL is an E3 ubiquitin ligase that |
| | | complexes with Cullin4 and DDB1, |
| | | and promotes p21 degradation after |
| | | UV damage. |
| E6-AP | p53, Dlg | E6-AP is also known as UBE3A. E6- |
| | | AP is a HECT domain E3 ubiquitin |
| | | ligase that interacts with Hepatitis C |
| | | virus (HCV) core protein and targets |
| | | it for degradation. The HCV core |
| | | protein is central to packaging viral |
| | | DNA and other cellular processes. |
| | | E6-AP also interacts with the E6 |
| | | protein of the human papillomavirus |
| | | types 16 and 18, and targets the p53 |
| | | tumor-suppressor protein for |
| | | degradation. |
| HACE1 | | HACE1 is an E3 ubiquitin ligase and |
| | | tumor suppressor. Aberrant |
| | | methylation of HACE1 is frequently |
| | | found in Wilms' tumors and |
| | | colorectal cancer. |
| HECTD1 | | HECTD1 is an ubiquitin E3 ligase |
| | | required for neural tube closure and |
| | | normal development of the |
| | | mesenchyme. |
| HECTD2 | | HECTD2 is a probable E3 ubiquitin |
| | | ligase and may act as a |
| | | succeptibility gene for |
| | | neurodegeneration and prion disease. |
| HECTD3 | | HECTD3 is a probable E3 ubiquitin |
| | | ligase and may play a role in |
| | | cytoskeletal regulation, actin |
| | | remodeling, and vesicle trafficking. |
| HECW1 | DVL1, | HECW1 is also known as NEDL1. |
| | mutant | HECW1 interacts with p53 and the |
| | SOD1, p53 | Wnt signaling protein DVL1, and |
| | | may play a role in p53-mediated cell |
| | | death in neurons. |
| HECW2 | p73 | HECW2 is also known as NEDL2. |
| | | HECW2 ubiquitinates p73, which is a |
| | | p53 family member. Ubiquitination |
| | | of p73 increases protein stability. |
| HERC2 | RNF8 | HERC2 belongs to a family of E3 |
| | | ubiquitin ligases involved in |
| | | membrane trafficking events. HERC2 |
| | | plays a role in the DNA damage |
| | | response through interaction with |
| | | RNF8. |
| HERC3 | | HERC3 belongs to a family of E3 |
| | | ubiquitin ligases involved in |
| | | membrane trafficking events. HERC3 |
| | | interacts with hPLIC-1 and hPLIC-2 |
| | | and localizes to the late endosomes |
| | | and lysosomes. |
| HERC4 | | HERC4 belongs to a family of E3 |
| | | ubiquitin ligases involved in |
| | | membrane trafficking events. HERC4 |
| | | is highly expressed in testis and may |
| | | play a role in spermatogenesis. |
| HERC5 | | HERC5 belongs to a family of E3 |
| | | ubiquitin ligases involved in |
| | | membrane trafficking events. HERC5 |
| | | is induced by interferon and other |
| | | pro-inflammatory cytokines and plays |
| | | a role in interferon-induced ISG15 |
| | | conjugation during the innate immune |
| | | response. |
| HUWE1 | N-Myc, | HUWE1 is also known as Mule. |
| | C-Myc, p53, | HUWE1 is a HECT domain E3 |
| | Mcl-1, | ubiquitin ligase that regulates |
| | TopBP1 | degradation of Mcl-1 and therefore |
| | | regulates DNA damage-induced apoptosis. |
| | | HUWE1 also controls neuronal |
| | | differentiation by destabilizing N-Myc, |
| | | and regulates p53-dependent and |
| | | independent tumor suppression via ARF. |
| HYD | CHK2 | HYD is also known as EDD or UBR5. |
| | | HYD is a regulator of the DNA damage |
| | | response and is overexpressed in many |
| | | forms of cancer. |
| ITCH | MKK4, RIP2, | ITCH plays a role in T cell receptor |
| | Foxp3 | activation and signaling through |
| | | ubiquitination of multiple proteins |
| | | including MKK4, RIP2 and Foxp3. |
| | | Loss of ITCH function leads to an |
| | | abberrant immune response and T |
| | | helper cell differentiation. |
| LNX1 | NUMB | LNX1 is an E3 ubiquitin ligase that |
| | | plays a role in cell fate determination |
| | | during embryogenesis through regulation |
| | | of NUMB, the negative regulator of |
| | | Notch signaling. |
| mahogunin | | Mahogunin is an E3 ubiquitin ligase |
| | | involved in melanocortin signaling. |
| | | Loss of mahogunin function leads to |
| | | neurodegeneration and loss of |
| | | pigmentation, and may be the |
| | | mechanism of action in prion disease. |
| MARCH-I | HLA-DRβ | MARCH1 is an E3 ubiquitin ligase |
| | | found on antigen presenting cells |
| | | (APCs). MARCH1 ubiquitinates MHC |
| | | class II proteins and downregulates |
| | | their cell surface expression. |
| MARCH-II | | MARCH-II is a member of the MARCH |
| | | family of E3 ubiquitin ligases. It |
| | | associates with syntaxin6 in the |
| | | endosomes and helps to regulate |
| | | vesicle trafficking. |
| MARCH-III | | MARCH-III is a member of the MARCH |
| | | family of E3 ubiquitin ligases. |
| | | MARCH-III associates with syntaxin6 |
| | | in the endosomes and helps to |
| | | regulate vesicle trafficking. |
| MARCH-IV | MHC class I | MARCH-IV is a member of the MARCH |
| | | family of E3 ubiquitin ligases. |
| | | MARCH-IV ubiquitinates MHC class I |
| | | proteins and downregulates their |
| | | cell surface expression. |
| MARCH-VI | | MARCH-VI is also known as TEB4 and |
| | | is a member of the MARCH family of |
| | | E3 ubiquitin ligases. It localizes |
| | | to the endoplasmic reticulum and |
| | | participates in ER-associated protein |
| | | degradation. |
| MARCH-VII | gp190 | MARCH-VII is also known as axotrophin. |
| | | MARCH-VII was originally identified |
| | | as a neural stem cell gene, but has |
| | | since been shown to play a role in |
| | | LIF signaling in T lymphocytes |
| | | through degradation of the LIF- |
| | | receptor subunit, gp190. |
| MARCH- | B7-2, MHC | MARCH-VIII is also known as c-MIR. |
| VIII | class II | MARCH-VIII causes the ubiquitination/ |
| | | degradation of B7-2, which is a co- |
| | | stimulatory molecule for antigen |
| | | presentation. MARCH-VIII has also |
| | | been shown to ubiquitinate MHC class |
| | | II proteins. |
| MARCH-X | | MARCH-X is also known as RNF190. |
| | | MARCH-X is a member of the MARCH |
| | | family of E3 ubiquitin ligases. |
| | | The putative role of MARCH-X is |
| | | not currently known. |
| MDM2 | p53 | MDM2, an E3 ubiquitin ligase for |
| | | p53, plays a central role in regulation |
| | | of the stability of p53. Akt-mediated |
| | | phosphorylation of MDM2 at Ser166 |
| | | and Ser186 increases its interaction |
| | | with p300, allowing MDM2-mediated |
| | | ubiquitination and degradation of |
| | | p53. |
| MEKK1 | c-Jun, Erk | MEKK1 is a well known protein |
| | | kinase of the STE11 family. MEKK1 |
| | | phosphorylates and activates |
| | | MKK4/7, which in turn activates |
| | | JNK1/2/3. MEKK1 contains a RING |
| | | finger domain and exhibits E3 |
| | | ubiquitin ligase activity toward c- |
| | | Jun and Erk. |
| MIB1 | Delta, Jagged | Mindbomb homolog 1 (MIB1) is an |
| | | E3 ligase that facilitates the |
| | | ubiquitination and subsequent |
| | | endocytosis of the Notch ligands, |
| | | Delta and Jagged. |
| MIB2 | Delta, Jagged | Mind Bomb 2 (MIB2) is an E3 ligase |
| | | that positively regulates Notch |
| | | Signaling. MIB2 has been shown to |
| | | play a role in myotube differentiation |
| | | and muscle stability. MIB2 |
| | | ubiquitinates NMDAR subunits to |
| | | help regulate synaptic plasticity in |
| | | neurons. |
| MycBP2 | Fbxo45, | MycBP2 is an E3 ubiquitin ligase |
| | TSC2 | also known as PAM. MycBP2 associates |
| | | with Fbxo45 to play a role in neuronal |
| | | development. MycBP2 also regulates the |
| | | mTOR pathway through ubiquitination |
| | | of TSC2. |
| NEDD4 | | NEDD4 is an E3 ubiquitin ligase |
| | | highly expressed in the early mouse |
| | | embryonic central nervous system. |
| | | NEDD4 downregulates both neuronal |
| | | voltage-gated Na+ channels (NaVs) |
| | | and epithelial Na+ channels (ENaCs) |
| | | in response to increased intracellular |
| | | Na+ concentrations. |
| NEDD4L | Smad2 | NEDD4L is an E3 ubiquitin ligase |
| | | highly expressed in the early mouse |
| | | embryonic central nervous system. |
| | | NEDD4L has been shown to negatively |
| | | regulate TGF-β signaling by |
| | | targeting Smad2 for degradation. |
| Parkin | | Parkin is an E3 ubiquitin ligase that |
| | | has been shown to be a key regulator |
| | | of the autophagy pathway. Mutations |
| | | in Parkin can lead to Parkinson's |
| | | Disease. |
| PELI1 | TRIP, IRAK | PELI1 is an E3 ubiquitin ligase that |
| | | plays a role in Toll-like Receptor |
| | | (TLR3 and TLR4) signaling to NF- |
| | | κB via the TRIP adaptor protein. |
| | | PELI1 has also been shown to |
| | | ubiquitinate IRAK. |
| Pirh2 | TP53 | Pirh2 is also known as RCHY1. Pirh2 |
| | | is a RING domain E3 ubiquitin |
| | | ligase. Pirh2 binds p53 and promotes |
| | | proteosomal degradation of p53 |
| | | independent of MDM2. Pirh2 gene |
| | | expression is controlled by p53, |
| | | making this interaction part of an |
| | | autoinhibitory feedback loop. |
| PJA1 | ELF | PJA1 is also known as PRAJA. PJA1 |
| | | plays a role in downregulating TGF-β |
| | | signaling in gastric cancer via |
| | | ubiquitination of the SMAD4 adaptor |
| | | protein ELF. |
| PJA2 | | PJA2 is an E3 ubiquitin ligase found |
| | | in neuronal synapses. The exact role |
| | | and substrates of PJA2 are unclear. |
| RFFL | p53 | RFFL is also known as CARP2 and is |
| | | an E3 ubiquitin ligase that inhibits |
| | | endosome recycling. RFFL also |
| | | degrades p53 through stabilization of |
| | | MDM2. |
| RFWD2 | MTA1, p53, | RFWD2 is also known as COP1. |
| | FoxO1 | RFWD2 is an E3 ubiquitin ligase that |
| | | ubiquitinates several proteins |
| | | involved in the DNA damage |
| | | response and apoptosis including |
| | | MTA1, p53, and FoxO1. |
| Rictor | SGK1 | Rictor interacts with Cullin1-Rbx1 to |
| | | form an E3 ubiquitin ligase complex, |
| | | and promotes ubiquitination and |
| | | degradation of SGK1. |
| RNF5 | JAMP, | RNF5 is also known as RMA5. RNF5 |
| | paxillin | plays a role in ER-associated |
| | | degradation of misfolded proteins and |
| | | ER stress response through |
| | | ubiquitination of JAMP. RNF5 also |
| | | plays a role in cell motility and has |
| | | been shown to ubiquitinate paxillin. |
| RNF8 | H2A, H2AX | RNF8 is a RING domain E3 ubiquitin |
| | | ligase that plays a role in the |
| | | repair of damaged chromosomes. RNF8 |
| | | ubiquitinates Histone H2A and H2A.X |
| | | at double-strand breaks (DSBs) which |
| | | recruits 53BP1 and BRCA1 repair |
| | | proteins. |
| RNF19 | SOD1 | RNF19 is also known as Dorfin. |
| | | Accumulation and aggregation of |
| | | mutant SOD1 leads to ALS disease. |
| | | RNF19 ubiquitinates mutant SOD1 |
| | | protein, causing a decrease in |
| | | neurotoxicity. |
| RNF190 | | see MARCH-X |
| RNF20 | Histone H2B | RNF20 is also known as BRE1. |
| | | RNF20 is an E3 ubiquitin ligase that |
| | | monoubiquitinates Histone H2B. |
| | | H2B ubiquitination is associated with |
| | | areas of active transcription. |
| RNF34 | Caspase- | RNF34 is also known as RFI. RNF34 |
| | 8, -10 | inhibits death receptor mediated |
| | | apoptosis through ubiquitination/ |
| | | degradation of caspase-8 and -10. |
| RNF40 | Histone H2B | RNF40 is also known as BRE1-B. |
| | | RNF40 forms a protein complex with |
| | | RNF20 resulting in the ubiquitination |
| | | of Histone H2B. H2B ubiquitination |
| | | is associated with areas of active |
| | | transcription. |
| RNF125 | | RNF125 is also known as TRAC-1. |
| | | RNF125 has been shown to positively |
| | | regulate T cell activation. |
| RNF128 | | RNF128 is also known as GRAIL. |
| | | RNF128 promotes T cell anergy and |
| | | may play a role in actin cytoskeletal |
| | | organization in T cell/APC |
| | | interactions. |
| RNF138 | TCF/LEF | RNF138 is also known as NARF. |
| | | RNF138 is associated with Nemo-like |
| | | Kinase (NLK) and suppresses Wnt/β- |
| | | Catenin signaling through |
| | | ubiquitination/degradation of |
| | | TCF/LEF. |
| RNF168 | H2A, H2A.X | RNF168 is an E3 ubiquitin ligase that |
| | | helps protect genome integrity by |
| | | working together with RNF8 to |
| | | ubiquitinate Histone H2A and H2A.X |
| | | at DNA double-strand breaks (DSB). |
| SCF/β- | IκBα, Wee1, | SCF/β-TrCP is an E3 ubiquitin ligase |
| TrCP | Cdc25A, β- | complex composed of SCF (SKP1- |
| | Catenin | CUL1-F-box protein) and the substrate |
| | | recognition component, β-TrCP (also |
| | | known as BTRC). SCF/β-TrCP mediates |
| | | the ubiquitination of proteins involved |
| | | in cell cycle progression, signal |
| | | transduction, and transcription. SCF/ |
| | | β-TrCP also regulates the stability |
| | | of β-catenin and participates in |
| | | Wnt signaling. |
| SCF/FBW7 | Cyclin E, c- | SCF/FBW7 is an E3 ubiquitin ligase |
| | Myc, c-Jun | complex composed of SCF |
| | | (SKP1-CUL1-F-box protein) and |
| | | the substrate recognition component, |
| | | FBW7. SCF/FBW7 mediates the |
| | | ubiquitination of proteins involved |
| | | in cell cycle progression, signal |
| | | transduction, and transcription. |
| | | Target proteins for SCF/FBW7 |
| | | include the phosphorylated forms of |
| | | c-Myc, Cyclin E, Notch intracellular |
| | | domain (NICD), and c-Jun. Defects |
| | | in FBXW7 may be a cause of breast |
| | | cancer. |
| SCF/Skp2 | p27, p21, | SCF/Skp2 is an E3 ubiquitin ligase |
| | Fox01 | complex composed of SCF |
| | | (SKP1-CUL1-F-box protein) and |
| | | the substrate recognition component, |
| | | Skp2. SCF/Skp2 mediates the |
| | | ubiquitination of proteins |
| | | involved in cell cycle progression |
| | | (specifically the G1/S transition), |
| | | signal transduction and transcription. |
| | | Target proteins for SCF/Skp2 include |
| | | the phosphorylated forms of p27Kip1, |
| | | p21Waf1/Cip1, and FoxO1. |
| SHPRH | PCNA | SHPRH is an E3 ubiquitin ligase that |
| | | plays a role in DNA replication |
| | | through ubiquitination of PCNA. |
| | | PCNA ubiquitination prevents |
| | | genomic instability from stalled |
| | | replication forks after DNA damage. |
| SIAH1 | β-catenin, | SIAH1 is an E3 ubiquitin ligase that |
| | Bim, TRB3 | plays a role in inhibition of Wnt |
| | | signaling through ubiquitination of |
| | | β-catenin. SIAH1 has also been |
| | | shown to promote apoptosis through |
| | | upregulation of Bim, and to |
| | | ubiquitinate the signaling adaptor |
| | | protein TRB3. |
| SIAH2 | HIPK2, | SIAH2 is an E3 ubiquitin ligase that |
| | PHD1/3 | plays a role in hypoxia through |
| | | ubiquitination and degradation of |
| | | HIPK2. SIAH2 also ubiquitinates |
| | | PHD1/3, which regulates levels of |
| | | HIF-1α in response to hypoxia. |
| SMURF1 | Smads | SMURF1 is an E3 ubiquitin ligase |
| | | that interacts with BMP pathway |
| | | Smad effectors, leading to Smad |
| | | protein ubiquitination and |
| | | degradation. Smurf1 negatively |
| | | regulates osteoblast differentiation |
| | | and bone formation in vivo. |
| SMURF2 | Smads, Mad2 | SMURF2 is an E3 ubiquitin ligase |
| | | that interacts with Smads from both |
| | | the BMP and TGF-β pathways. |
| | | SMURF2 also regulates the mitotic |
| | | spindle checkpoint through |
| | | ubiquitination of Mad2. |
| TOPORS | p53, NKX3.1 | TOPORS is an E3 ubiquitin ligase |
| | | and a SUMO ligase. TOPORS |
| | | ubiquitinates and sumoylates p53, |
| | | which regulates p53 stability. |
| | | TOPORS has also been shown to |
| | | ubiquitinate the tumor supressor |
| | | NKX3.1. |
| TRAF6 | NEMO, Akt1 | TRAF6 is an E3 ubiquitin ligase that |
| | | functions as an adaptor protein in IL- |
| | | 1R, CD40, and TLR signaling. |
| | | TRAF6 promotes NF-κB signaling |
| | | through K63 polyubiquitination of |
| | | IKK, resulting in IKK activation. |
| | | TRAF6 has also been shown to |
| | | ubiquitinate Akt1, causing its |
| | | translocation to the cell membrane. |
| TRAF7 | | TRAF7 is an E3 ubiquitin ligase and |
| | | SUMO ligase that functions as an |
| | | adaptor protein in TNF Receptor and |
| | | TLR signaling. TRAF7 has been |
| | | shown to be capable of self- |
| | | ubiquitination and plays a role in |
| | | apoptosis via MEKK3-mediated |
| | | activation of NF-κB. |
| TRIM63 | Troponin I, | TRIM63 is also known as Murf-1. |
| | MyBP-C, | TRIM63 is a muscle-specific E3 |
| | MyLC1/2 | ubiquitin ligase whose expression is |
| | | upregulated during muscle atrophy. |
| | | TRIM63 has been shown to |
| | | ubiquitinate several important muscle |
| | | proteins including troponin I, MyBP- |
| | | C, and MyLC1/2. |
| UBE3B | | UBE3B is an E3 ubiquitin ligase |
| | | identified through sequence analysis. |
| | | The specific substrates and cellular |
| | | function of UBE3B is currently |
| | | unknown. |
| UBE3C | | UBE3C is an E3 ubiquitin ligase also |
| | | known as KIAA10. UBE3C is highly |
| | | expressed in muscle and may interact |
| | | with the transcriptional regulator |
| | | TIP120B. |
| UBR1 | | UBR1 is an E3 ubiquitin ligase |
| | | responsible for proteasomal |
| | | degradation of misfolded cytoplasmic |
| | | proteins. UBR1 has also been shown |
| | | to be a ubiquitin ligase of the N-end |
| | | rule proteolytic pathway, which |
| | | regulates degradation of short-lived |
| | | proteins. |
| UBR2 | Histone H2A | UBR2 is an E3 ubiquitin ligase that |
| | | has been shown to ubiquitinate |
| | | histone H2A, resulting in |
| | | transcriptional silencing. UBR2 is |
| | | also part of the N-end rule proteolytic |
| | | pathway. |
| UHRF2 | PCNP | UHRF2 is also known as NIRF. |
| | | UHRF2 is a nuclear protein that may |
| | | regulate cell cycle progression |
| | | through association with Chk2. |
| | | UHRF2 also ubiquitinates PCNP and |
| | | has been shown to play a role in |
| | | degradation of nuclear aggregates |
| | | containing polyglutamine repeats. |
| VHL | HIF-1α | VHL is the substrate recognition |
| | | component of the ECV (Elongin B/C, |
| | | Cullen-2, VHL) E3 ubiquitin ligase |
| | | complex responsible for degradation |
| | | of the transcription factor HIF-1α. |
| | | Ubiquitination and degradation of |
| | | HIF-1α takes place only during |
| | | periods of normoxia, but not during |
| | | hypoxia, thereby playing a central |
| | | role in the regulation of gene |
| | | expression by oxygen. |
| WWP1 | ErbB4 | WWP1 is an E3 ubiquitin ligase |
| | | commonly found to be overexpressed |
| | | in breast cancer. WWP1 has been |
| | | shown to ubiquitinate and degrade |
| | | ErbB4. Interestingly, the WWP1 |
| | | homolog in C. elegans was found to |
| | | increase life expectancy in response |
| | | to dietary restriction. |
| WWP2 | Oct-4 | WWP2 is an E3 ubiquitin ligase that |
| | | has been shown to ubiquitinate/ |
| | | degrade the stem cell pluripotency |
| | | factor Oct-4. WWP2 also ubiquitinates |
| | | the transcription factor EGR2 to |
| | | inhibit activation-induced T cell death. |
| ZNRF1 | | ZNRF1 is an E3 ubiquitin ligase |
| | | highly expressed in neuronal cells. |
| | | ZNRF1 is found in synaptic vesicle |
| | | membranes and may regulate |
| | | neuronal transmissions and plasticity. |

Substrates

Examples of substrates include polypeptides that are known to be ubiquitinated in vivo in humans by E3 ligase or any other ubiquitinating enzyme.

According to a specific embodiment, the substrate is one that is known to be ubiquitinated differentially in a disease such as cancer.

Exemplary substrates that may be expressed in the bacteria have been described herein above.

According to a particular embodiment, the substrate is selected from the group consisting of IKZF1, IKZF2, IKZF3, CK1 alpha, β-catenin, c-myc, k-ras, PHD3, SPROUTY2, Mitofusin 1, 2, MIRO, NEMO, SMADs, TβR-I, P53, S5A, HHR23, EPHEXIN5, ARC, PPARα, cyclin-B, Cdc25C and Calmodulin.

It will be appreciated that as well as expressing the substrate and the ubiquitin (together with the split reporter polypeptide), the recombinant bacteria should also express the ubiquitinating or deubiquitinating enzyme.

In one embodiment, the bacteria express at least one E1 enzyme, at least one E2 conjugating enzyme and preferably at least one E3 enzyme.

Preferably, the bacteria expresses the E2 conjugating enzyme that is a cognate pair for the E3 enzyme.

In another embodiment, the bacteria expresses at least one deubiquitinating enzyme.

As mentioned, following expression of the components in the bacterial cell, the candidate agent (or library of agents) is contacted with the cell.

Contacting said bacterial cells with the agent (or library of agents) can be performed by any in vitro conditions including for example, adding the agent to cells such that the agent is in direct contact with the cells. According to some embodiments of the invention, the cells are incubated with the agent. The conditions used for incubating the cells are selected for a time period/concentration of cells/concentration of agent/ratio between cells and agent and the like which enable the agent to bind to its targets.

Measuring the level or the rate of accumulation of said detectable or selectable signal is effected according to the detectable/selectable signal used. Thus, for example if the detectable signal is a fluorescent signal, the amount of detectable signal or rate of accumulation of detectable signal may be measured using a fluorescent microscope/detector. If the detectable signal is a selectable signal, the amount of detectable signal or rate of accumulation of detectable signal may be measured by counting the number of cells present in the presence of the antibiotic (e.g. by analyzing OD600 nm). A change (e.g. increase) in the level of detectable signal (or an increase in the number of cells that are resistant to the effects of an antibiotic) as compared to the level in the absence of the agent, is indicative of an agent which mediates the binding of the first polypeptide to the second polypeptide.

The increase in detectable signal is typically about 10%, e.g., higher than about 20%, e.g., higher than about 30%, e.g., higher than about 40%, e.g., higher than about 50%, e.g. higher than about 60%, higher than about 70%, higher than about 80%, higher than about 90%, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times of at least one reference cell (e.g., the identical bacterial cell in the absence of the agent). The upregulation in the detectable signal can be also determined using logarithmic fold changes.

According to another aspect of the present invention there is provided a method of identifying an agent which mediates the binding of a first polypeptide to a second polypeptide comprising:

(a) contacting a bacterial cell with the agent, wherein the bacterial cell outputs a detectable or selectable signal which correlates with the non-direct binding of the first polypeptide to said second polypeptide via said agent; and

(b) measuring the level or the rate of accumulation of said detectable or selectable signal, wherein a change in the level as compared to the level in the absence of said agent, is indicative of an agent which mediates the binding of the first polypeptide to said second polypeptide, wherein:

(i) the first polypeptide is a substrate for ubiquitination;

(ii) said second polypeptide is attached to ubiquitin;

(iii) said bacterial cell expresses a ubiquitinating enzyme which attaches said ubiquitin to said first polypeptide if said agent mediates the binding of said first polypeptide to said second polypeptide.

This aspect of the present invention is exemplified in FIGS. 1 and 2. In this aspect, the second polypeptide is not a ubiquitnating enzyme. In a further embodiment, the first polypeptide is also not a FIGS. 1 and 2. In this aspect, the second polypeptide is not a ubiquitnating enzyme. Agents which are screened according to this aspect of the present invention are typically bifunctional agents as further described herein above.

Examples of polypeptides which are substrates for ubiquitination are described herein above.

Ubiquitinating enzymes are also further described herein above.

Measuring the level or the rate of accumulation of the detectable or selectable signal is described herein above.

According to still another aspect of the present invention there is provided a method of identifying an agent which mediates the binding of an E2 conjugating enzyme to an E3 ligase comprising:

(a) contacting a bacterial cell which expresses said E2 conjugating enzyme and said E3 ligase with the agent, wherein the bacterial cell outputs a detectable or selectable signal which correlates with the non-direct binding of the E2 conjugating enzyme to said E3 ligase via said agent, wherein said non-direct binding of said E2 conjugating enzyme to said E3 ligase via said agent brings about the ubiquitination of a protein of interest; and

(b) measuring the level or the rate of accumulation of said detectable or selectable signal, wherein a change in the level as compared to the level in the absence of said agent, is indicative of an agent which mediates the binding of said E2 conjugating enzyme to said E3 ligase.

Exemplary agents which may be screened for enhancing the binding of E3 ligase to E2 conjugating enzyme are provided herein above. According to a particular embodiment, the agent is a drug candidate for treating cancer (e.g. breast cancer or ovarian cancer).

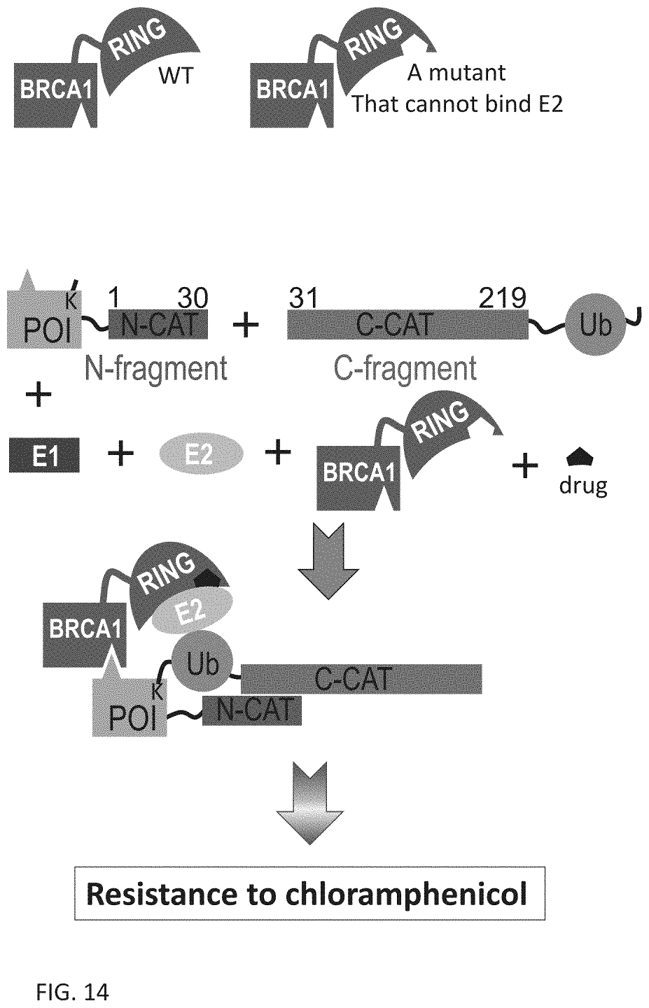

Exemplary E2 conjugating enzymes and E3 ligases are listed herein above. According to a specific embodiment the E3 ligase is BRCA1. The present inventors contemplate analyzing BRCA1 which comprise mutations abolish or decrease E2 interaction—see for example, FIG. 14.

The present inventors also contemplate the use of kits for carrying out the screening.

According to one aspect of the present invention the kit comprises:

(i) a first polynucleotide which encodes a first polypeptide fragment which is operably linked to a bacterial regulatory sequence, and a first cloning site, wherein a position of said first cloning site is selected such that upon insertion of a sequence which encodes a first test polypeptide into said first cloning site, following expression in a bacterial cell, a fusion protein is generated which comprises said first test polypeptide in frame with said first polypeptide fragment; and

(ii) a second polynucleotide comprising a second nucleic acid sequence encoding a second polypeptide fragment which is attached to ubiquitin, the second nucleic acid sequence being operably linked to a bacterial regulatory sequence, the second nucleic acid sequence comprising a second cloning site, wherein a position of said second cloning site is selected such that upon insertion of a sequence which encodes a second test polypeptide into said second cloning site, following expression in a bacterial cell, a fusion protein is generated which comprises said second test polypeptide in frame with said second polypeptide fragment;

wherein said first polypeptide fragment associates with said second polypeptide fragment to generate a reporter polypeptide dependent on ubiquitination of said test polypeptide.

The term “cloning site” refers to a location on a vector into which DNA can be inserted. The term “multiple cloning site” or “mcs” refers to a synthetic DNA sequence that contains any one or a number of different restriction enzyme sites to permit insertion at a defined locus (the restriction site) on a vector. The term “unique cloning site” refers to a cloning site that appears one time with a given DNA sequence.

In one embodiment, the regulatory sequence is a promoter, examples of which are provided herein above.

The first polynucleotide of this aspect of the present invention comprises a first protein fragment of a split protein reporter and the second polynucleotide of this aspect of the present invention comprises the second protein fragment of the split protein reporter. Examples of split protein reporters are described herein above.

It will be appreciated that the polynucleotides of this aspect of the invention are such that at least two fusion proteins may be expressed therefrom—a first one comprising a first test polypeptide in frame with a first protein fragment of a split protein reporter; and a second comprising a second test polypeptide in frame with a second protein fragment of a split protein reporter.

As used herein, the phrase “in frame” refers to the expression of a functional single protein comprising two components. The two components of the protein may be linked directly or may be linked via a linking peptide.

The kits of this aspect of the present invention may comprise additional components for carrying out the screening assays described herein. In one embodiment, the kits comprise the mediating agents which are screened for activity. The mediating agents of this aspect of the present invention are typically heterobifunctional, comprising a ligand to a first target protein, a ligand to a second target protein (e.g. an E3 ubiquitin ligase) and optionally a linker connecting the two ligands, as further described herein above. The ligands to the first and second target protein are typically not identical.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, biophysical, bacterial genetics and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al.

PROteolysis TArgeting Chimeras (PROTACs) are biforked molecules that recruit an E3 ligase to a neo target protein to promote its ubiquitylation and degradation.

An example of a PROTAC is Papilloma virus E6 which functions as a biforked protein that associates P53 with E6AP, a Ub E3-ligase that promotes the ubiquitylation and degradation of P53 hence leading to cervical cancer. Thalidomide and thalidomide-like molecules including lenalidomide and pomalidomide (collectively termed immunomodulatory drugs; IMiDs) have therapeutic efficacy in multiple myeloma and other cancers. Recently, using immobilized IMiDs in combination with SILAC (stable isotope labeling of amino acids in cell culture) based quantitative mass spectrometry, it was demonstrated that the CRBN (a CRL) E3-ligase binds these molecules. Moreover, it has been demonstrated that IMiDs function as glue that bring neo-targets to the ligase. Specifically, it was demonstrated that thalidomide-like molecules target the CRL4CRBN and promote the ubiquitylation of the IKAROS family transcription factors IKZF1 and IKZF3, which have a strong impact on cancer development.

The present inventors propose that bacterial-based selection systems for analyzing ubiquitination can be used to facilitate the discovery of PROTACs and other drug-dependent protein-protein interactions (PPIs).

FIG. 1 shows that two proteins that do not interact in the absence of a mediator, may form a novel interaction interface in the presence of a small molecule. One difficulty in identifying such small molecules is that the approach requires a combinatorial screen as the molecule should simultaneously interacts with ‘A’ and ‘B’ proteins. Although binding of the low affinity mediator should in principal be sufficient to promote growth in the presence of the antibiotic, the probability of identifying such biforked molecules is very low. To circumvent this challenge, the present inventors propose screening in two steps, where they will use a biforked molecule that one side is known to interact with a given protein. This embodiment is illustrated in FIG. 2. Here, a known molecule (black pentagon) that binds protein ‘A’ is chemically tethered to a library of small molecules. This new chimera library is then used to screen to identify novel molecules that interacts with protein ‘B’.

Importantly, replacing the small molecule library with a protein library instead of ‘B’ can identify all the proteins that bind a specific molecule. This may be important for diagnosis and prediction of sides effect of given molecule or to identify the protein interactors of biological small molecules.

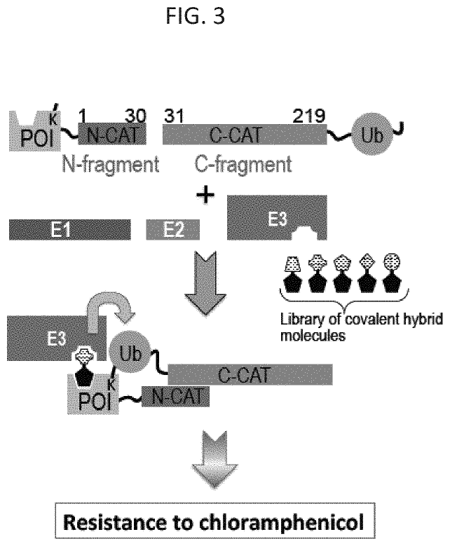

The selection system illustrated in FIG. 3 can be employed in order to identify novel PROTAC molecules for specific E3-ligase and specific neo-ubiquitylation-targets.

Here a library of biforked molecules is generated where the head of the chimera is known to bind a specific protein of interest and the tail of the chimera (which binds to the E3 ligase) is not known. Importantly, one can switch the known and the unknown interactions: where the tail of the chimeric bioforked molecule which binds the ligase is known and the head of the chimera that binds the ubiquitylation target (POI) is not known. Using these two steps one can construct a new PROTAC for selected (given) E3-ligase and neo-target(s).

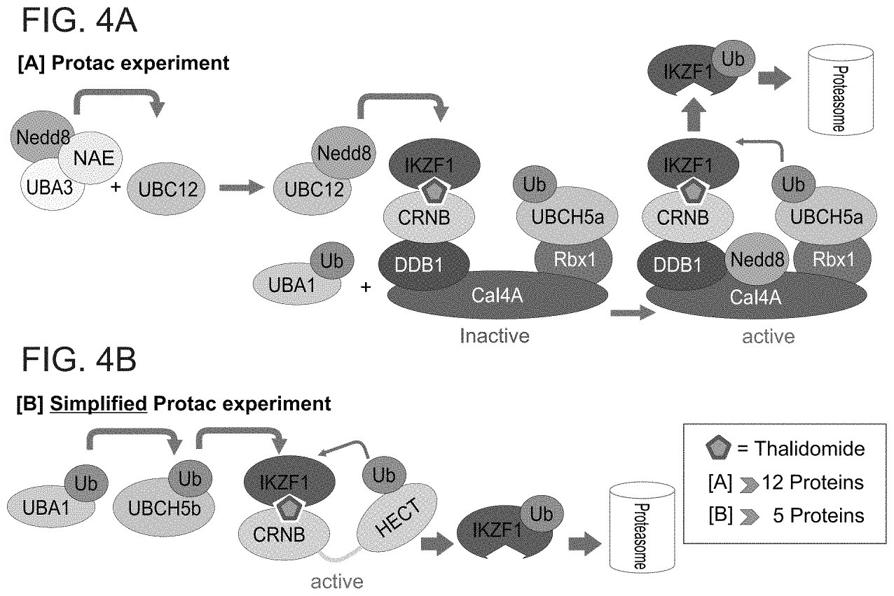

The use of CRL (Cullin-Rbx-ligase) E3s to identify neo-targets in this context is not trivial as it requires the simultaneous expression of 12 eukaryotic proteins (see FIGS. 4A-B) in E. coli including Ub and target fused to the split antibiotic resistance protein, E1, E2, Cullin, Rbx, Skip, F-box and the Nedd8ylation system including Nedd8, NEA, UBA3, Ubc12.

The present inventors propose to bypass this challenge by generating a fusion protein which comprises a CRBN domain that binds to the bioforked molecule, but which in itself has no E3 ligase activity, and a E3 ligase activity domain from another E3 ligase (such as HECT, RING or RBR)—see FIG. 5.

Materials and Methods

Construction of split-CAT vectors: Split CAT-Selection system: The pnCAT and pcCAT vectors were prepared by complete chemical synthesis assembly (Gibson et al, Nature Methods volume 6, pages 343-345 (2009)). The pnCAT is based on a modified pCD-Sub vector. The cDHFR-linker2 was removed from the pCDSub01 vector by complete chemical synthesis assembly (Gibson et al, Nature Methods volume 6, pages 343-345 (2009)). Then, the cDNA of the nCAT fragment (residues 1-30) was PCR-amplified with a primer containing sequence coding to GSG linker and sub-cloned downstream and in frame to the POI. The pcCAT is based on a modified pND-Ub vector. The nDHFR fragment in the pND-Ub vector was replicated with cCAT (residues 31-218).

Activity assays with split-CAT system: Vectors containing the split-CAT ubiquitylation system were co-transformed into E. coli and grew in the presence of the relevant antibiotics (e.g. kanamycin and streptomycin). Overnight culture was harvested and the media was replaced with fresh LB containing 8 μg/ml chloramphenicol. The culture was diluted to O.D.600=0.2 and dispended to a 96 or 384 well plate supplemented with potential small molecule drugs or DMSO only. The plate was placed in a shaker/incubator/reader and OD600 was continuously monitored during the growth.

This procedure was used to read the ubiquitylation dependent growth of IKZF1, IKZF3, CK1a and GSPT1.

Dose response experiments were performed as described above with increased concentration of the studied drug such as TLD/LLD or CC-885.

Results

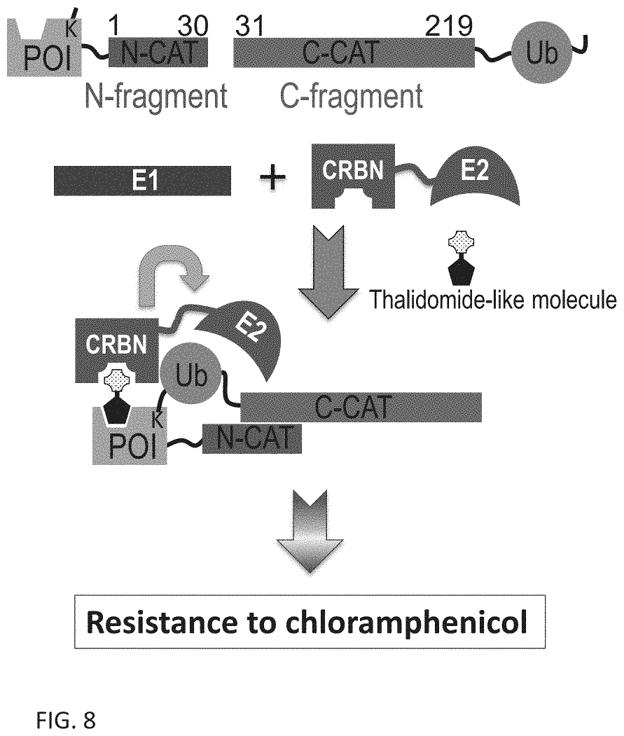

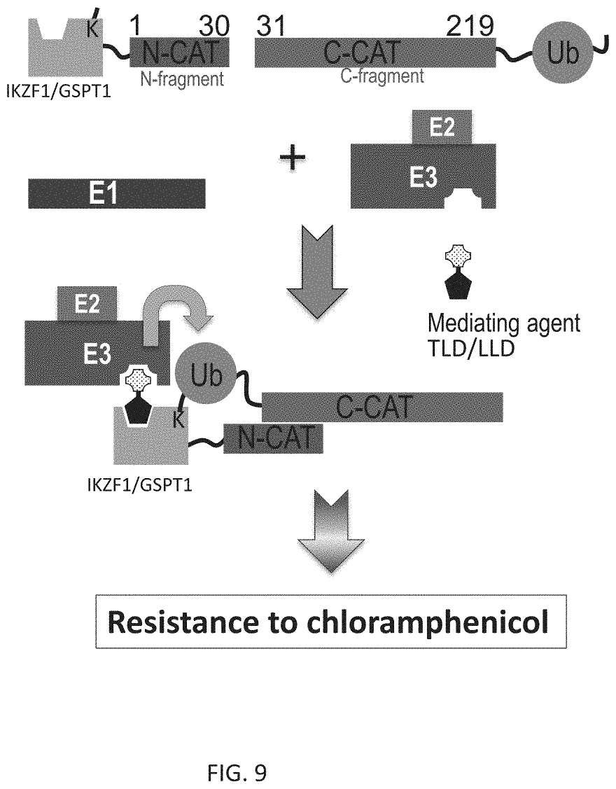

In a first experiment, IKZF1 (Q13422) and GSPT1 (P15170) were analyzed as potential ubiquitylation targets. They were expressed in-frame upstream to the N-CAT fragment as shown in FIG. 9. These targets were coexpressed with C-CAT-Ub and E1 and the Cereblon-E2 (Ubc4) fusion. As seen in FIGS. 10A-C, Thalidomide and Lenalidomide (TLD/LLD) significantly promoted the growth of E. coli that express IKZF1 but not GSTP1 (negative control). These assays were performed in 96 and 384 well plates. GSPT1 was chosen as negative control as it has been described to undergo CRBN dependent ubiquitylation in the presence of CC-885 but not TLD or LLD [Matyskiela et al. Nature 535, 252-257 (2016)].

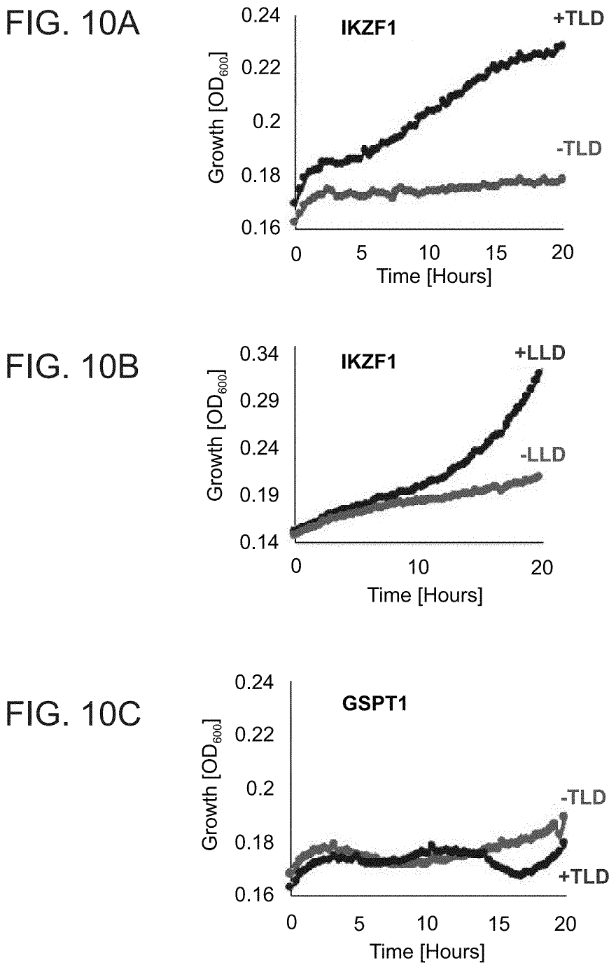

One well known characteristic of a PROTAC assay is its optimum (bell-shape) response curve. This is in sharp contrast to inhibitors or activators that typically show saturation sigmoid response curves. This unique characteristic is due to the dual affinity of the PROTAC molecule to the ligase and the target. To demonstrate that the split-CAT selection system functions due to PROTAC activity, dose response assays were performed (FIGS. 11A-C). These results demonstrate that the selection system maintains this well-known optimum characteristic response to TLD and LLD.

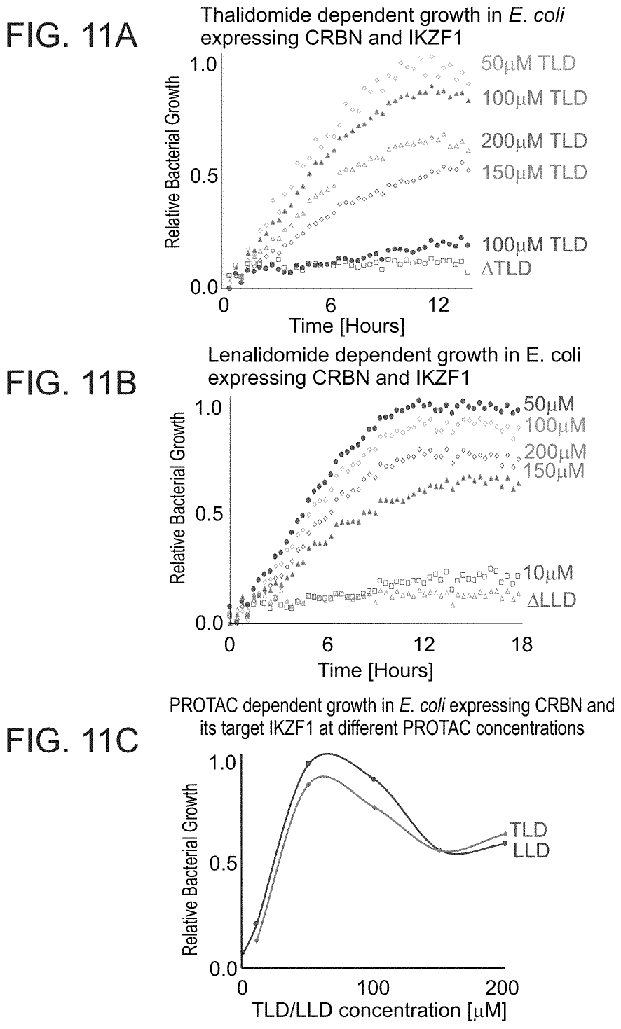

In a second experiment, CK1a (P48729) was analyzed as a potential ubiquitylation target. It was expressed in-frame upstream to the N-CAT fragment. The target was co-expressed with C-CAT-Ub and E1 and the Cereblon-E2 (Ubc4) fusion. As seen in FIGS. 12A-C, Thalidomide and Lenalidomide (TLD/LLD) significantly promoted the growth of E. coli that express CK1a. These assays were performed in 96 and 384 well plates.

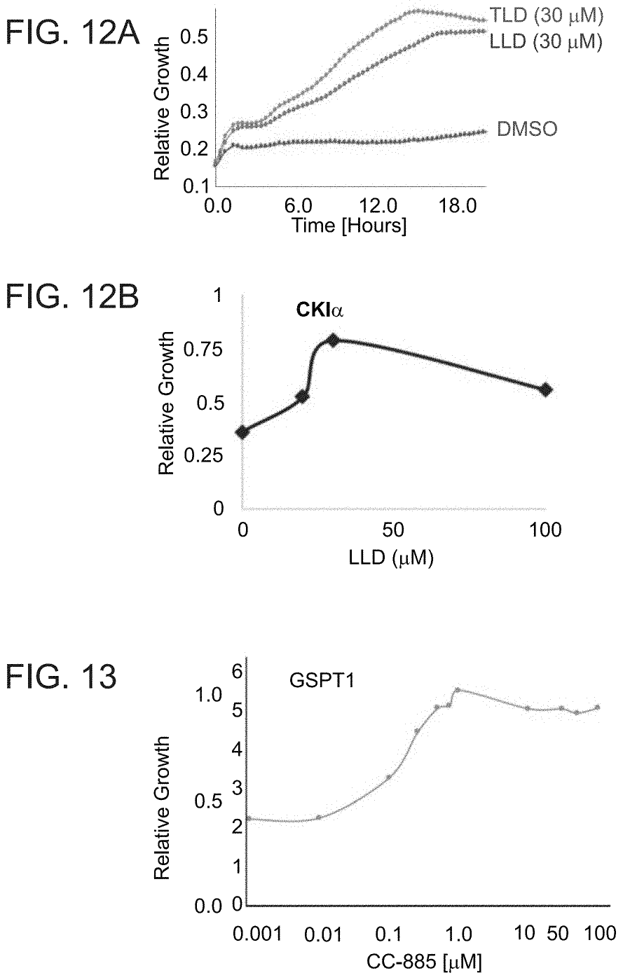

In a third experiment, GSPT1 was analyzed as a potential ubiquitylation target. It was expressed in-frame upstream to the N-CAT fragment. The target was co-expressed with C-CAT-Ub, wheat E1 and Cereblon-E2 (Ubc4) fusion. As seen in FIG. 13, CC-885 significantly promoted the growth of E. coli that express GSPT1. These assays were performed in 96 and 384 well plates.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, the priority document of this application is hereby incorporated herein by reference in its entirety.

1. A method of identifying an agent which mediates the binding of a first polypeptide to a second polypeptide comprising:
(a) contacting a bacterial cell which expresses said first polypeptide and said second polypeptide with the agent, wherein the bacterial cell outputs a detectable or selectable signal which correlates with the non-direct binding of the first polypeptide to said second polypeptide via said agent, wherein said non-direct binding of said first polypeptide to said second polypeptide via said agent brings about the ubiquitination of said first polypeptide or said second polypeptide; and
(b) measuring the level or the rate of accumulation of said detectable or selectable signal, wherein a change in the level as compared to the level in the absence of said agent, is indicative of an agent which mediates the binding of said first polypeptide to said second polypeptide.

2. The method of claim 1, wherein said first polypeptide is a ubiquitin E3 ligase.

3. The method of claim 2, wherein said ubiquitin E3 ligase is a chimeric E3 ligase.

4. The method of claim 1, wherein said second polypeptide is a substrate for said ubiquitin E3 ligase.

5. The method of claim 1, wherein said first polypeptide is attached to ubiquitin.

6. The method of claim 5, wherein said second polypeptide is not a ubiquitin E3 ligase.

7. The method of claim 6, wherein the bacterial cell expresses at least one ubiquitin ligase.

8. The method of claim 7, wherein said ubiquitin ligase comprises ubiquitin E3-ligase.

9-10. (canceled)

11. The method of claim 1, wherein said agent is not ubiquitin.

12. The method of claim 1, wherein said agent is a small molecule agent.

13. The method of claim 1, wherein the bacterial cell further expresses an E1 activating enzyme and/or an E2 conjugating enzyme.

14. The method of claim 1, wherein said selectable signal is a selectable polypeptide.

15. The method of claim 14, wherein said selectable polypeptide is a split antibiotic resistance polypeptide.

16-20. (canceled)

21. A method of identifying an agent which mediates the binding of a first polypeptide to a second polypeptide comprising:
(a) contacting a bacterial cell with the agent, wherein the bacterial cell outputs a detectable or selectable signal which correlates with the non-direct binding of the first polypeptide to said second polypeptide via said agent; and
(b) measuring the level or the rate of accumulation of said detectable or selectable signal, wherein a change in the level as compared to the level in the absence of said agent, is indicative of an agent which mediates the binding of the first polypeptide to said second polypeptide, wherein:
(i) the first polypeptide is a substrate for ubiquitination;
(ii) said second polypeptide is attached to ubiquitin;
(iii) said bacterial cell expresses a ubiquitinating enzyme which attaches said ubiquitin to said first polypeptide if said agent mediates the binding of said first polypeptide to said second polypeptide.

22. The method of claim 21, wherein said ubiquitinating enzyme is a ubiquitin E3-ligase.

23-24. (canceled)

25. The method of claim 21, wherein said second polypeptide is not a ubiquitinating enzyme.

26. The method of claim 21, wherein said agent is not ubiquitin.

27. The method of claim 21, wherein said agent is a small molecule agent.

28. The method of claim 22, wherein said ubiquitinating enzyme further comprises an E1 activating enzyme and an E2 conjugating enzyme.

29-35. (canceled)

36. A kit comprising:
(i) a first polynucleotide which encodes a first polypeptide fragment which is operably linked to a bacterial regulatory sequence, and a first cloning site, wherein a position of said first cloning site is selected such that upon insertion of a sequence which encodes a first test polypeptide into said first cloning site, following expression in a bacterial cell, a fusion protein is generated which comprises said first test polypeptide in frame with said first polypeptide fragment; and
(ii) a second polynucleotide comprising a second nucleic acid sequence encoding a second polypeptide fragment which is attached to ubiquitin, the second nucleic acid sequence being operably linked to a bacterial regulatory sequence, the second nucleic acid sequence comprising a second cloning site, wherein a position of said second cloning site is selected such that upon insertion of a sequence which encodes a second test polypeptide into said second cloning site, following expression in a bacterial cell, a fusion protein is generated which comprises said second test polypeptide in frame with said second polypeptide fragment;
wherein said first polypeptide fragment associates with said second polypeptide fragment to generate a reporter polypeptide dependent on ubiquitination of said first test polypeptide.

37. A method of identifying an agent which mediates the binding of a ubiquitin E3 ligase to a test polypeptide comprising:
(a) contacting a bacterial cell which expresses said E3 ligase with the agent, wherein the bacterial cell outputs a detectable or selectable signal which correlates with the non-direct binding of the E3 ligase to said test polypeptide via said agent; and
(b) measuring the level or the rate of accumulation of said detectable or selectable signal, wherein a change in the level as compared to the level in the absence of said agent, is indicative of an agent which mediates the binding of the E3 ligase to the test polypeptide.

38. (canceled)

39. The method of claim 37, wherein said ubiquitin E3-ligase is a chimeric ubiquitin E3-ligase.

40. (canceled)

41. The method of claim 37, wherein the bacterial cell further expresses an E1 activating enzyme and an E2 conjugating enzyme.

42. The method of claim 37, wherein the bacterial cell expresses:
(a) ubiquitin attached to a first polypeptide fragment; and
(b) said test polypeptide attached to a second polypeptide fragment, wherein said first polypeptide fragment associates with said second polypeptide fragment to generate a reporter polypeptide on ubiquitination of said substrate.

43-50. (canceled)

51. The method of claim 37, wherein said agent comprises a glutarimide ring.

52. The method of claim 51, wherein said agent is a thalidomide.

53. A kit comprising:
(i) a first polynucleotide which encodes a first polypeptide fragment which is operably linked to a bacterial regulatory sequence, and a cloning site, wherein a position of said cloning site is selected such that upon insertion of a sequence which encodes a test polypeptide into said cloning site, following expression in a bacterial cell, a fusion protein is generated which comprises said test polypeptide in frame with said first polypeptide fragment;
(ii) a second polynucleotide comprising a second nucleic acid sequence encoding a second polypeptide fragment which is attached to ubiquitin, the second nucleic acid sequence being operably linked to a bacterial regulatory sequence, wherein said first polypeptide fragment associates with said second polypeptide fragment via a mediating agent to generate a reporter polypeptide dependent on ubiquitination of said test polypeptide; and
(iii) said mediating agent.

54. A method of identifying an agent which mediates the binding of an E2 conjugating enzyme to an E3 ligase comprising:
(a) contacting a bacterial cell which expresses said E2 conjugating enzyme and said E3 ligase with the agent, wherein the bacterial cell outputs a detectable or selectable signal which correlates with the non-direct binding of the E2 conjugating enzyme to said E3 ligase via said agent, wherein said non-direct binding of said E2 conjugating enzyme to said E3 ligase via said agent brings about the ubiquitination of a protein of interest; and
(b) measuring the level or the rate of accumulation of said detectable or selectable signal, wherein a change in the level as compared to the level in the absence of said agent, is indicative of an agent which mediates the binding of said E2 conjugating enzyme to said E3 ligase.


SEQUENCE LISTING (11 sequences)

SEQ ID NO: 1 - protein, 219 aa, Escherichia coli
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 2 - protein, 30 aa, Artificial sequence
  CAT N terminal protein sequence
MEKKITGYTTVDISQWHRKEHFEAFQSVAQ

SEQ ID NO: 3 - protein, 190 aa, Artificial sequence
  CAT C  terminal protein sequence
MCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 4 - DNA, 93 bp, Artificial sequence
  CAT N terminal DNA sequence
atggagaaaaaaatcactggatataccaccgttgatatatcccaatggcatcgtaaagaacattttgaggcatttcagtcagttgctcaataa

SEQ ID NO: 5 - DNA, 573 bp, Artificial sequence
  CAT C terminal DNA sequence
atgtgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 6 - protein, 28 aa, Artificial sequence
  CAT N terminal protein sequence
MEKKITGYTTVDISQWHRKEHFEAFQSV

SEQ ID NO: 7 - protein, 192 aa, Artificial sequence
  CAT C  terminal protein sequence
MAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 8 - DNA, 660 bp, Escherichia coli
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 9 - protein, 76 aa, Artificial sequence [sequence omitted]

SEQ ID NO: 10 - protein, 75 aa, Artificial sequence
  yeast  ubiquitin protein sequence
MQIFVKTLTGKTITLEVESSDTIDNVKSKIQDKEGIPPDQQRLIFAGKQLEDGRLSDYNIQKESTLHLVLRLRGG

SEQ ID NO: 11 - protein, 18 aa, Artificial Sequence
  Amino acid sequence of the active site of CAT
AFQSVAQCTYNQTVQLDI